(19) United States
(12) Patent Application Publication
(10) Pub. No.: US 2010/0304461 A1
(43) Pub. Date: Dec. 2, 2010

(54) Portable, Temperature and Chemically Inducible Expression Vector for High Cell Density Expression of Heterologous Genes in Escherichia Coli

(76) Inventors: John W. Brandis, Austin, TX (US); Kenneth A. Johnson, Austin, TX (US)

Correspondence Address: BAKER BOTTS L.L.P.;PATENT DEPARTMENT, 98 SAN JACINTO BLVD., SUITE 1500, AUSTIN, TX, 78701-4039, US

(21) Appl. No.: 12/664,118

(22) PCT Filed: Jun. 12, 2008
(86) PCT No.: PCT/US08/66742

§ 371 Date: Dec. 11, 2009

Related U.S. Application Data
(60) Provisional application No. 60/943,507, filed on Jun. 12, 2007.

(51) Int. Cl. C12N 9/12; C07H 21/04; C12N 15/63; C12N 1/00; C12N 1/21

(52) U.S. Cl. 435/194; 536/23.1; 435/320.1; 435/243; 435/252.33

(57) ABSTRACT

The present disclosure relates to nucleic acids comprising a sequence of SEQ ID NO: 1. The nucleic acid may be an isolated DNA and/or may be in the form of a plasmid or an expression vector. It may also be comprised in a microorganism. The nucleic acid may further comprise sequences that encode a protein. The self-replicating expression plasmid comprising a DNA sequence of the disclosure may be used to produce one or more protein. The production of one or more protein by a plasmid of the disclosure may be controlled by temperature and/or chemical induction. The disclosure also provides methods of obtaining high yields of proteins and methods for purifying such proteins, such as the LdK39 protein or a fragment thereof.

### RELATED APPLICATION

This application is a U.S. national stage application of International Application No. PCT/US2008/066742 filed Jun. 12, 2008, which designates the United States of America, and claims the benefit of U.S. Provisional Patent Application Ser. No. 60/943,507, filed Jun. 12, 2007, the entire disclosure of which is hereby incorporated by reference.

### TECHNICAL FIELD OF THE DISCLOSURE

The present disclosure relates to recombinant DNA molecules encoding plasmids in Escherichia coli, including a new inducible expression plasmid and methods for protein production as well as protein purification of a protein expressed by an expression plasmid of the disclosure (e.g. the large fragment of Thermus aquaticus DNA polymerase I).

### BACKGROUND OF THE DISCLOSURE

Enzyme structure and function studies require increasingly large amounts of pure enzymes. For example, to crystallize more complicated structures such as a DNA polymerase in a ternary complex with DNA plus an in-coming nucleotide, multi-milligram quantities of the enzyme are necessary to define and to optimize crystallization strategies, or to measure individual steps in an enzyme reaction pathway, transient kinetic methods require that the enzyme be present in reagent concentrations. It is common for research enzymology labs to use recombinant DNA technology to produce workable amounts of enzymes typically using Escherichia coli (E. coli) because it is inexpensive and easy to culture in shake-flasks. In addition, over the course of the past two decades much attention has been focused on strong promoter systems to improve heterologous gene expression in E. coli. High yields have been reported for many different enzymes but this usually refers to a high yield per cell in relatively low cell density cultures. Overall yields per culture batch or cycle were typically a few to tens of milligrams which were sufficient in most cases for starting crystallization efforts or for several kinetic experiments. The production of hundreds of milligram quantities of an enzyme using E. coli usually requires fermentation technology, equipment, and methods such as stirred fermenters with nutrient feeding capabilities that are unavailable to the average enzymology laboratory that must rely, instead on floor model gyratory shaker-incubators.

Existing expression vector systems based upon the strong and tightly controllable promoters from bacteriophage, e.g., phage lambda, have been widely used for high specific cell yields of recombinant products. These vectors are typically controlled by the temperature-sensitive lambda repressor gene, λcI857, that may be located in the host chromosome, on an accessory plasmid, or on-board the expression vector itself. While popular, cI857-controlled expression vectors can only be induced by a temperature jump typically requiring a rapid temperature increase from a non-permissive 32° C. to 42° C. to inactivate the repressor. Rapid temperature jumps are, however, difficult to accomplish in multi-vessel, shaker-incubators.

### SUMMARY OF THE DISCLOSURE

The present disclosure provides, in some embodiments, a high copy number expression plasmid, that is may be inducible by chemical induction and/or temperature induction or both, that may have a moderate to high cell density capability in shake-flasks, may have host strain “portability” and may provide high yield of recombinant products.

In some embodiments, a vector of the disclosure may comprise a promoter, e.g. a powerful rightward promoter from bacteriophage lambda, cloned into the high copy-number plasmid, pUC19. This promoter/copy-number combination may provide high levels of transcription following induction. The promoter/gene transcriptional unit may be separated from the plasmid origin of replication by the T1T2 transcription terminators from the rrnB operon of E. coli thus preventing post-induction transcription from interfering with plasmid replication/stability. Expression may be controlled by a modified lambda repressor gene, λcI^{ts} ind⁺, “on-board” the plasmid thus making it possible to rapidly screen a variety of host strains to optimize expression yields, stability, and the solubility of recombinant products. This repressor may allow use of chemical or temperature induction or both in recA⁺ strains which may be more robust than typical recA⁻ cloning hosts. The disclosure describes, in one example, use of a plasmid, pcI^{ts} ind⁺, to express a modified version of the large fragment of Taq DNA polymerase I, as a test enzyme, using all three modes of induction, chemical alone, temperature alone, or both, in shake-flasks routinely achieving final cell densities of 9 to 12 A₆₀₀/ml and yields of purified enzyme in the range of 30 to 35 mg/liter of culture and 100 to 300 mg per batch.

In some embodiments, the compositions, systems and methods disclosure relates to an isolated DNA comprising a sequence of SEQ ID NO: 1. The disclosure provides a recombinant plasmid comprising an isolated DNA comprising a sequence of SEQ ID NO: 1. In some embodiments, the plasmid is a vector. The vector may be a cloning vector and/or an expression vector. The disclosure also related to a microorganism comprising DNA comprising a sequence of SEQ ID NO: 1.

In some embodiments, the disclosure relates to a self-replicating nucleic acid molecule comprising: a promoter; at least one inducible repressor; a high copy number origin of replication; a sequence able to prevent transcription from the promoters from entering the region comprising the origin of replication; and a multiple cloning site wherein at least one nucleic acid encoding a protein of interest may be cloned. The promoter may be a promoter of the bacteriophage lambda and may be exemplified in non-limiting embodiments by the rightward promoter of bacteriophage lambda or the leftward promoter of bacteriophage lambda.

In some embodiments, the compositions, systems and methods of the disclosure relate to inducible repressor may be a temperature-inducible repressor. In some embodiments, the inducible repressor is a chemically-inducible repressor. The inducible repressor may be a temperature and chemically-inducible repressor. For example, a temperature and chemically-inducible repressor may be a lambda repressor λcI^{ts} ind⁺. In some embodiments, the promoter is controlled by the repressor.

The disclosure also provides methods of producing at least one protein, comprising inducing expression of the at least one protein using a recombinant plasmid comprising an isolated DNA having a sequence of SEQ ID NO: 1, wherein inducing comprises temperature induction. In some embodiments, inducing further comprises chemical induction. The recombinant plasmid comprising an isolated DNA having a sequence of SEQ ID NO: 1 may further comprises at least one nucleic acid encoding the at least one protein that is being produced by the method.

In some embodiments, methods of the disclosure relate to of producing at least one protein, comprising inducing expression of the at least one protein using a recombinant plasmid comprising an isolated DNA having a sequence of SEQ ID NO: 1, wherein inducing comprises chemical induction. The inducing may further comprises temperature induction.

The disclosure also relates to protein production systems comprising a self-replicating nucleic acid molecule comprising: a promoter of bacteriophage lambda; a high copy number origin of replication; a sequence able to prevent transcription from said promoters from entering the region comprising the origin of replication; and a multiple cloning site; and an inducible repressor located on a chromosome.

In some embodiments, the self-replicating nucleic acid molecule and the repressor may be located in a living organism. In some embodiments, the repressor may be located on a host chromosome in the living organism.

In some embodiments, a protein production system is provided comprising a self-replicating nucleic acid molecule comprising: a promoter of bacteriophage lambda; a high copy number origin of replication; a sequence able to prevent transcription from said promoters from entering the region comprising the origin of replication; a multiple cloning site; and an inducible repressor.

The disclosure also relates to methods for protein purification comprising: a) obtaining a cell lysate from a cell comprising DNA having a sequence of SEQ ID NO: 1; b) treating the cell lysate with heat to denature cellular proteins; c) precipitating and removing cellular DNA thereby obtaining a supernatant comprising the denatured cellular proteins; d) applying the supernatant on a system of two chromatography columns, the first column comprising a cation-exchanger and the second column comprising an affinity-chromatography column; and eluting the proteins, thereby obtaining purified proteins. In some examples, the method may be used with the protein production system of the disclosure. Thereby proteins that are produced using the inducible, high-copy number expression plasmids of the disclosure may be purified. In some embodiments, the purification methods are rapid and efficient.

In one embodiment, which may use materials and methods of the embodiments described above, an E. coli-based protein production system is provided. The system may include an E. coli cell having a self-replicating nucleic acid molecule. The self-replicating nucleic acid molecule may include: a promoter of bacteriophage lambda, a high copy number origin of replication, a sequence able to prevent transcription from said promoters from entering the region comprising the origin of replication, and a sequence encoding an LdK39 protein or fragment thereof.

### BRIEF DESCRIPTION OF THE DRAWINGS

Some specific example embodiments of the disclosure may be understood by referring, in part, to the following description and the accompanying drawings, wherein:

FIG. 1 shows a diagram depicting a partial restriction site map for pcI^{ts},ind⁺ modKlenTaqI showing the restriction sites used for the insertion of the modified KlenTaq I gene, mKlenTaqI; as well as transcription terminators, T1T2; origin of replication, pUC19 ori; the β-lactamase gene, AMP; the lambda repressor, pcI^{ts},ind⁺; and the rightward promoter, λP_{R} in accord with one embodiment of the present disclosure;

FIG. 2 shows growth curves comparing the cell density of temperature-induced cells with chemically-induced cells over time in accordance with one embodiment of the present disclosure;

FIG. 3 depicts a comparison of protein yields for both temperature-induced cells and chemically-induced cells in accord with one embodiment of the present disclosure;

FIG. 4 depicts protein yields for cells that were both temperature- and chemically-induced in accordance with one embodiment of the present disclosure;

FIG. 5 shows a growth curve for large-scale shake-flask expression using chemical- and temperature-induction in accordance with one embodiment of the present disclosure;

FIG. 6 depicts protein yields for large-scale shake-flask expression using chemical and temperature induction in accordance with one embodiment of the present disclosure;

FIG. 7 depicts an elution profile where the major peak corresponds to purified modKlenTaq1 in accordance with one embodiment of the present disclosure;

FIG. 8 shows a gel analysis of the column fractions used in the preparation of FIG. 7 wherein 5 μl aliquots from peak column fractions were analyzed by 12% SDS-PAGE, in accordance with one embodiment of the present disclosure;

FIG. 9 shows a diagram depicting another partial restriction site map in accordance with one embodiment of the present disclosure;

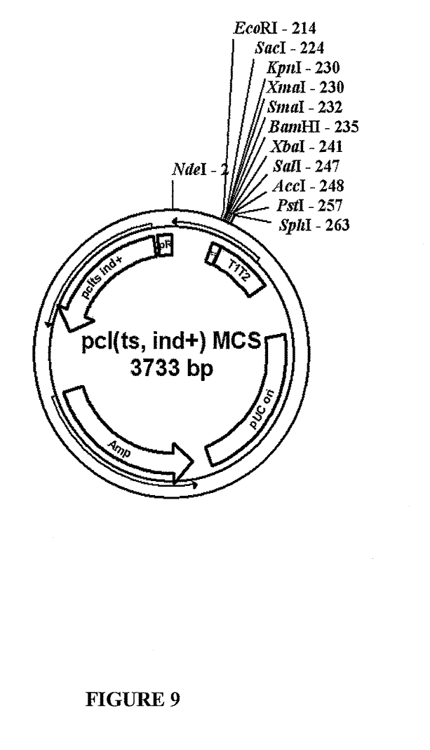

FIG. 10 shows a diagram of a partial restriction map of the Leishmania donovani kinesin 39 (LdK39) gene;

FIG. 11 shows an expression vector containing a portion of the LdK39 gene, according to an embodiment of the present disclosure;

FIG. 12 shows a growth curve for the vector of FIG. 11 in E. coli in small-scale shake-flask expression using chemical only- and chemical and temperature-induction in accordance with one embodiment of the present disclosure;

FIG. 13 depicts protein yields for small-scale shake-flask expression of the vector of FIG. 11 in E. coli using chemical and temperature induction in accordance with one embodiment of the present disclosure;

FIG. 14 depicts antibody detection of a Flag-tag added to the LdK39 protein as expressed in a vector similar to that of FIG. 11 (the vector of FIG. 11 with Flag sequences added) in E. coli.

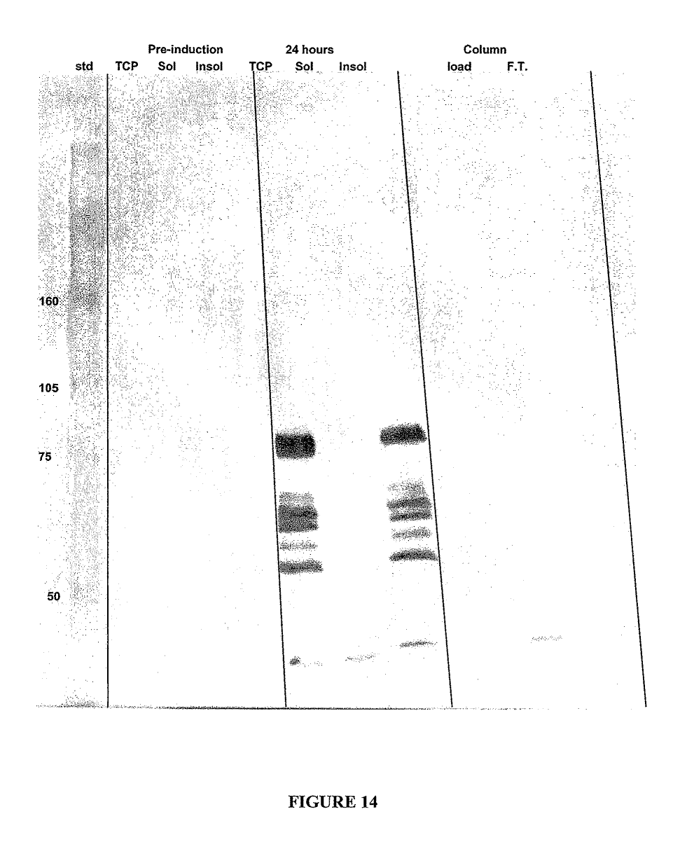

While the present disclosure is susceptible to various modifications and alternative forms, specific example embodiments thereof have been shown in the drawings and are herein described in detail. It should be understood, however, that the description herein of specific example embodiments is not intended to limit the disclosure to the particular forms disclosed herein, but on the contrary, this disclosure is to cover all modifications and equivalents.

### DETAILED DESCRIPTION

Current methods to produce useful amounts of enzymes or other proteins, such as immunogenic proteins may often be expensive, time consuming and/or require expensive laboratory equipment and expertise. New methods may contribute to inexpensive or easy production of useful amounts of enzymes or other proteins, such as immunogenic proteins and/or reduced costs. Embodiments of the present disclosure provide a system and method that remains simple while achieving increased yields and/or final cell densities when compared to alternative systems.

When used herein, the following abbreviations and/or acronyms indicated the terms identified below:

ATCC refers to American Type Culture Collection;

CV, column volume;

DNAP, DNA polymerase;

ΔΔ, heat-treated protein sample;

EDTA, ethylenediamine tetraacetic acid;

LB, Luria-Bertani medium;

LdK39, Leishmania donovani kinesin 39;

OD₆₀₀, optical density at 600 nm;

PAGE, polyacrylamide gel electrophoresis;

PCR, polymerase chain reaction;

PEI, polyethyleneimine;

PMSF, phenylmethane sulfonyl fluoride;

SDS, sodium dodecyl sulfate;

TBS, Terrific Broth plus Salts medium;

TCP, Total cell protein;

TRIS, tris hydroxymethylaminoethane; and

TYE, tryptone-yeast extract medium.

The present disclosure provides expression vectors and methods that may comprise the following characteristics: 1) chemical and/or temperature induction; 2) moderate to high cell density capability in shake-flasks; 3) host strain “portability;” and 4) high specific cell yield of one or more proteins that are being expressed. An expression vector of the disclosure may take advantage of the powerful rightward promoter from bacteriophage lambda cloned into a high copy-number plasmid, pUC19. This promoter/copy-number combination may provide high levels of transcription following induction. The promoter/gene transcriptional unit may be separated from the plasmid origin of replication by the T1T2 transcription terminators from the rrnB operon of E. coli thereby preventing post-induction transcription from interfering with plasmid replication/stability. Furthermore, transcription may be controlled by a modified lambda repressor “on-board” the plasmid allowing rapid screening of a variety of host strains to optimize expression yields, stability, and solubility of recombinant products. This repressor makes it possible to use either chemical or temperature induction or both in recA⁺ strains which may be far more robust than typical recA⁻ cloning hosts.

This disclosure describes methods using a plasmid, e.g. pcI^{ts} ind⁺, to express a modified version of the large fragment of Taq DNA polymerase I, as a test enzyme, using all three modes of induction, chemical alone, temperature alone, and both, in shake-flasks routinely achieving final cell densities of 9 to 12 A₆₀₀/ml and yields of purified enzyme in the range of 30 to 35 mg/liter of culture and 100 to 300 mg per batch. It should be noted, however, that persons having ordinary skill in the art will be able to apply the teachings of the present disclosure using additional test enzymes and with a wide range of results. One of skill in the art, in light of this disclosure, will also recognize that other promoters, origins of replication, transcription terminators, repressors, and the like may be used.

### Examples

Some specific embodiments of the disclosure may be understood, by referring, at least in part, to the following examples. These examples are not intended to represent all aspects of the disclosure in its entirety. Variations will be apparent to one skilled in the art. The examples described herein may describe techniques, materials, processes and/or other concepts used in at least one example of practice of the teachings of the present disclosure, but should, however, not be construed to limit the scope of the those teachings.

### Example 1

### Materials and Methods

### Materials

Bacteriophage lambda DNA, λcI857 ind 1 Sam7, pUC19 DNA, chemically competent E. coli C2984H cells (K12 F⁻ proA⁺B⁺ lacI^{q} Δ (lac-proAB) glnV zgb-210::Tn10(Tet^{R}) endA1 thi-1 Δ(hsdS-mcrB)5 recA⁺), and all restriction enzymes were obtained from New England Biolabs. DH5α (K12 F⁻ 80ΔlacZ M15(lacZYA-argF) U169 recA endA1 hsdR17(r_{K12}⁻m_{K12}⁻) phoA supE44 thi-1 gyrA96 relA1) chemically competent cells were purchased from Invitrogen. Thermus aquaticus YT-1 lyophilized cells (ATCC #25104) were obtained from the American Type Culture Collection and grown in Castenholtz 1% TYE medium at 70° C. Chromosomal DNA was isolated using the Genomic DNA Purification Protocol and columns from Qiagen Inc.

### Culture Media

Transformed E. coli cells were grown in TBS medium or on LB plates at appropriate temperatures as are known in the art. Ampicillin (100 μg/ml) was added as required for ampicillin selection. Thermus aquaticus YT-1 cells were grown in Castenholtz 1% TYE plus vitamins and salts as described in the ATCC literature (recipe #461) with gentle shaking at 70° C.

### Cloning Thermus Aquaticus DNA Polymerase I

The chromosomal DNA region spanning the DNA polymerase gene, Taq DNAP I, of Thermus aquaticus was isolated by PCR amplification using the DNAP I primers as shown in Table I and purified chromosomal DNA as template. The amplicon was cut with Bgl2 and Sph1 and subcloned into pUC19. The modified KlenTaq (“modKlenTaq1”) version of this polymerase gene was constructed by PCR amplification of the catalytic domain region using the modKlenTaq Primer and DNAP I Reverse Primer as shown in Table I. The forward primer adds an Nde1 site at the start of the coding region for the truncated version of the enzyme plus seven additional amino acids. The reverse primer adds an Sph1 site immediately adjacent to the stop codon. This amplicon was cut with Nde1 and Sph1 and subcloned into a modified pUC19 vector containing the T1T2 transcription terminator region from the rrnB operon of E. coli between the multi-cloning site and the origin of replication region in the plasmid. This formed the “base” plasmid which was used to construct the final expression vector by methods described below.

### Expression Vector Construction

The region of the lambda genome containing the repressor gene, cI857 ind 1, and the rightward promoter, λP_{R}, was isolated as a PCR amplicon spanning bases λ37151-λ38039 using the primers shown in Table I and purified lambda DNA as template. The reverse primer (λ37151) was designed to generate an Nde1 site at the original start codon for the λcro gene (“CATATG”). The forward primer (λ38039) was designed to add a Kas1 site 3′ to the λcI857 ind 1 gene.

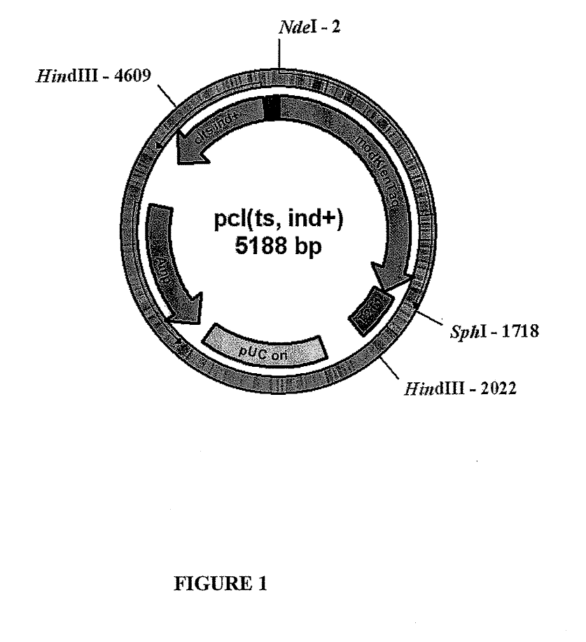

However, Kas1 digests of the amplicon generated a shorter than expected fragment indicating additional cutting within the coding region of the repressor gene. Therefore, the amplicon was cut with Mfe1 (originally at λ37186) plus Nde1 and subcloned into the “base” plasmid described above that was cut with EcoR1 and Nde1 generating pcI857^{ts} ind1-mKlenTaq1. The lambda repressor ind 1 mutation originally at position λ37589 was “back-mutated” to the wild-type sequence from T to C (with subsequent loss of the Hind3 site originally at λ37584) using site-directed mutagenesis forming the expression plasmid, pcI^{ts} ind⁺ modKlenTaqI as shown in FIG. 1. This plasmid was then used for expression testing.

### Expression Testing

The plasmid, pcI^{ts} ind⁺ modKlenTaqI, was transformed into chemically competent C2984H (recA⁺) or DH5α (recA⁻) cells, spread onto LB plus ampicillin plates, and incubated at 30° C. Ampicillin resistant colonies were selected and used to inoculate expression cultures in 75 ml TBS in 500-ml baffle-bottomed Erylenmeyer flasks shaken at 150 rpm at 30 or 32° C. When the cultures reached a cell density of 4 A₆₀₀/ml, the cells were induced by one of three methods: 1) chemical-induction which was achieved, in one example, by addition of nalidixic acid to about 50 μg/ml; 2) temperature-induction which was achieved, in one example, by rapidly changing the temperature to 42° C. by swirling flasks in a water bath and maintaining for 20 minutes after which incubation was continued at 37° C.; or 3) by both chemical- and temperature-induction, which was achieved, in one example, by adding nalidixic acid to the culture and the temperature setting was increased to 37° C. from a starting temperature of 32° C. or 30° C.

### Gel Samples

At appropriate times shown in the Figures, samples were removed from the cultures and placed on ice. Cells were pelleted at 6000×g for 5 minutes at room temperature. Cell pellets were resuspended in Lysis Buffer (50 mM TRIS, 2 mM EDTA, pH 8) plus lysozyme (0.5 mg/ml) and incubated at 37° C. for 10 minutes. Sodium chloride was added to the lysate to a final concentration of 500 mM to prevent the polymerase from binding to DNA in the pellets. After briefly sonicating the lysate to reduce viscosity, an aliquot was removed as the “Total Cell Protein Sample.” The remainder of the lysate was centrifuged at 13,000×g for 10 minutes at room temperature and an aliquot was removed from the supernatant to represent the “Soluble Protein Sample”. The remainder of the supernatant was heat treated at 75° C. for 45 minutes. Insoluble material was pelleted at 13,000×g for 5 minutes at room temperature and an aliquot was removed from the supernatant as the “Heat-treated Protein Sample.” Protein samples were analyzed by 8% SDS-PAGE. Protein concentrations were determined by Bradford assay (BioRad, Richmond, Calif.).

### Large-Scale Cultures

Six 2.8-liter baffle-bottomed Fernbach flasks (Bellco BioTech) each containing 1.5-liters of TBS and ampicillin were used to grow C2984H cells transformed with pcI^{ts} ind⁺ modKlenTaqI at 30° C. with shaking at 150 rpm. When the cultures reach cell densities above 3 OD₆₀₀/ml, the cultures were induced using temperature induction and chemical induction by either raising the shaker incubator temperature setting to 37° C. or by adding nalidixic acid to a final concentration of 50 mg/liter. Pre-induction and Harvest Samples were removed and processed as described above for SDS-PAGE. The cells were harvested at 24 hours post inoculation by centrifugation at 6,000×g for 20 minutes at 4° C. Cell pellets were weighed and stored at −20° C.

### Purification

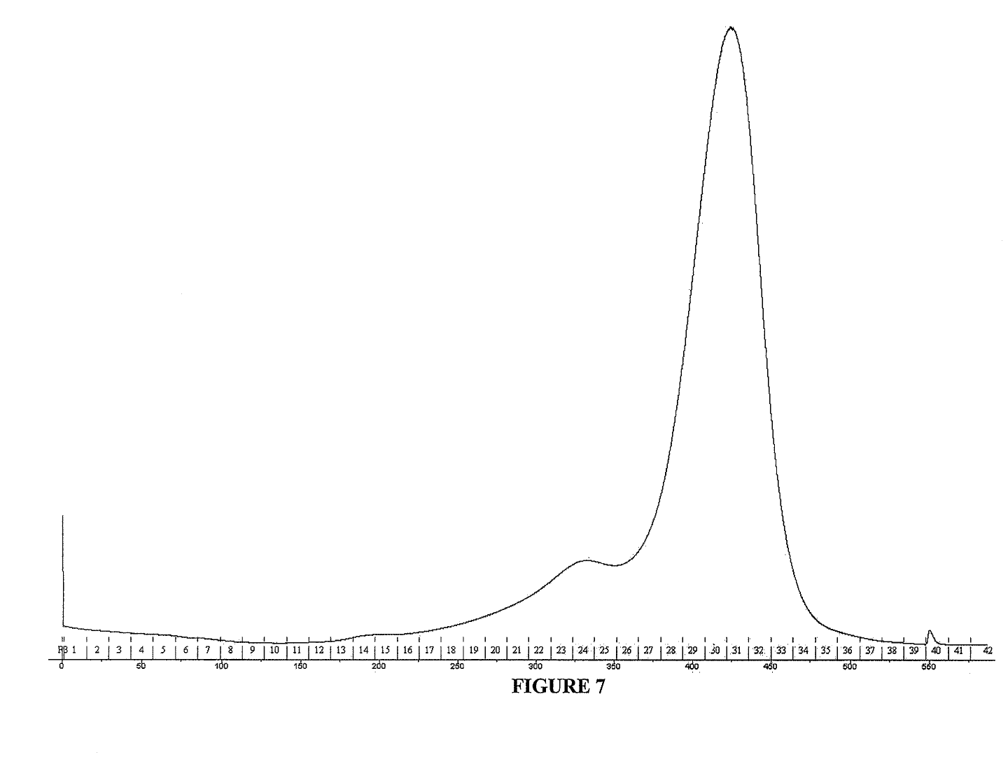

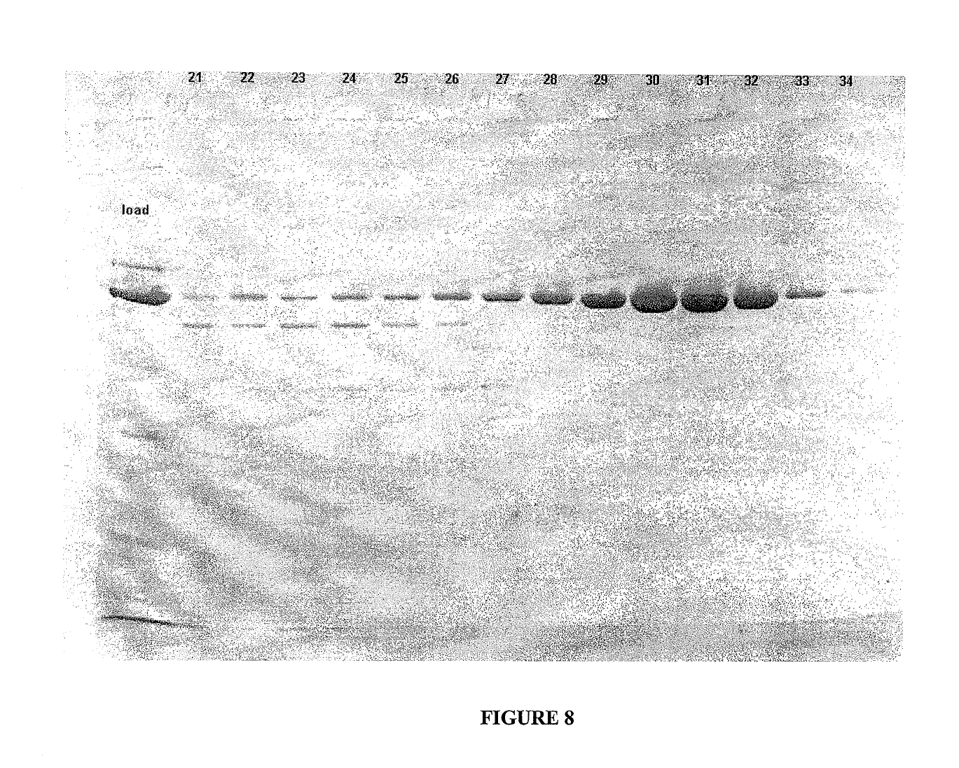

Frozen cell pastes were resuspended on ice in 5 volumes of Lysis Buffer (50 mM TRIS, 2 mM EDTA, 50 mM NaCl, 50 μM PMSF, pH 8) and lysozyme was added to 0.15 mg/ml. After 30 minutes, the lysate was sonicated to reduce viscosity. Sodium chloride was added to a final concentration of 0.25 M, and the sonicate was slowly added to an equal volume of Lysis Buffer in a water bath at 80° C. The temperature was kept above 60° C. during additions. After all the lysate was added, the mixture was incubated at 80° C. for an additional 45 minutes to precipitate host proteins. The heat treated lysate was cooled on ice and 10% polyethyleneimine was added to a final concentration of 0.3%. After 30 minutes, cell debris and denatured protein were pelleted at 10,000×g for 30 minutes at 4° C. The supernatant was diluted 3-fold with column buffer (20 mM TRIS, 1 mM EDTA, 0.05% TWEEN-20, 1% glycerol, pH 8.0) and loaded onto tandem BioRex-70 (2.6×20 cm) and Heparin-agarose (2.6×15 cm) columns. After washing with column buffer plus 100 mM NaCl until the OD₂₈₀ returned to background, modKlenTaq1 was eluted from the Heparin-agarose column using a 5.5 CV linear gradient (100 to 650 mM NaCl). The major peak eluting from the affinity column was modKlenTaq1 as shown in FIG. 7. Each fraction was around 14 ml. Aliquots from the fractions were analyzed by 12% SDS-PAGE as shown in FIG. 8. Peak fractions were pooled and flash frozen in liquid nitrogen, and stored at −80° C.

The examples resulting from at least one use of the process or materials described above were analyzed as described below. Although the results disclosed may be representative of the results expected when practicing the teachings disclosed herein, they should not be construed as limiting to the scope of the process. For instance, persons having ordinary skill in the art may be able to adjust process steps and/or constituents without departing from the scope of the present disclosure.

TABLE 1
Oligodeoxynucleotide Primers

| | |
|---|---|
| DNAP I Forward Primer | gcatcagaagctcAGATCTacctgcctgag |
| DNAP I Reverse Primer | cagcaataGCATGCtcactccttggcggagagcca |
| mod-KlenTaq Primer | cgatgaCATATGggtaaacgtaaatctactgcctttctggagaggct |
| lambda 37151 | agctctaaGGCGGCggagtgaaaattcccctaattcgatgaagattct |
| lambda 38039 | ttgatacCATATG aacctccttagtacatgcaaccatt |

Table 1 lists the primers used to construct and modify the expression plasmid, pcIts ind+ modKlenTaq1. Primers that have “cryptic” restriction sites to facilitate insertions are shown in CAPS. Underlined bases represent portions of coding regions for the genes indicated.

### Example 2

### Expression Plasmid Construction and Testing

The segment of phage lamdba genome spanning the λcI repressor, λO_{R} and λP_{R} region may be used for the design and construction of expression plasmids because it functions as a “self-contained” transcriptional control unit. The repressor protein may have very tight control over transcription from the rightward promoter. Using PCR primers containing cryptic restriction sites as shown in Table I and purified lambda DNA, an amplicon was generated that had modified ends for subcloning. By changing the bases just before the start codon of the λcro gene, a unique Nde1 site was introduced, which was used for the insertion of heterologous coding sequences.

FIG. 1 shows a partial restriction map for the plasmid, pcI^{ts} ind⁺ modKlenTaq1. The diagram shows the restriction sites used for the insertion of the modified KlenTaq I gene, mKlenTaq1, as well as transcription terminators, T1T2; the origin of replication, pUC19 ori; the β-lactamase gene, AMP; the lambda repressor, pcI^{ts} ind⁺; and, the rightward promoter, λP_{R}. The map shows that there are two Hind3 sites but only one site (equivalent to λ37459) in the repressor gene because the ind 1 to ind⁺ “back-mutation” eliminates the second site (equivalent to λ37589, T to C) that was originally in the λcI857 gene.

The transcriptional control unit consists of a fragment of the lambda genome spanning bases λ37187 to λ38043 as described above in Materials and Methods. The λcI857 ind 1 repressor originally has two Hind3 restriction sites at λ37584 and λ37459. The former site contains the ind 1 mutation that renders the repressor resistant to cleavage by RecA protein. Using site-directed mutagenesis, the final T of that Hind3 site was mutated to a C, eliminating the restriction site, and restoring sensitivity to RecA cleavage, the ind⁺ phenotype.

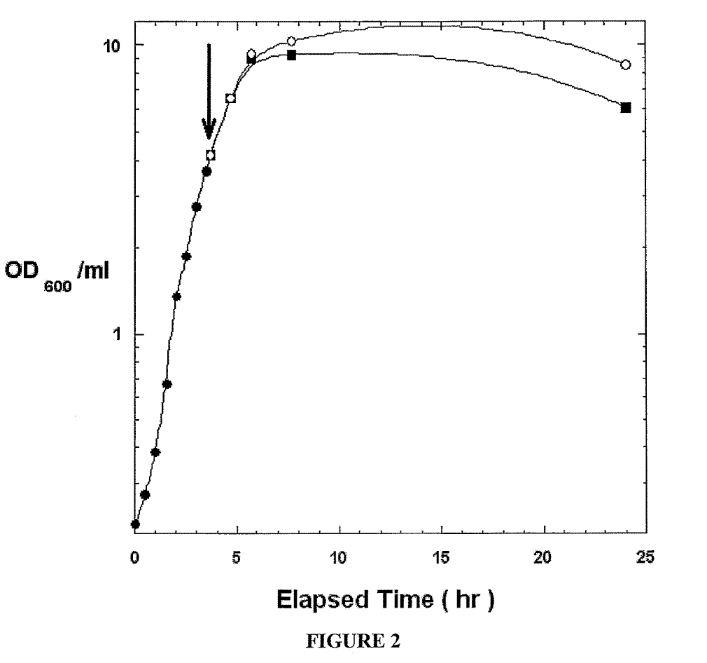

FIG. 2 shows growth curves comparing the cell density of temperature-induced cells versus chemically-induced cells over time in accordance with teachings of the present disclosure. An overnight culture of C2984H cells transformed with pcI^{ts} ind⁺ modKlenTaqI was used to inoculate 225 ml TBS plus ampicillin (100 μg/ml) and grown at 32° C. (solid circles in FIG. 2). At a cell density of 4 OD₆₀₀/ml (arrow), the culture was split into two subcultures: A) Chemical Induction Alone; solid squares (addition of nalidixic acid to 50 μg/ml and 30° C. for the duration of the experiment); and, B) Temperature Induction Alone; open circles (swirling in a 42° C. water bath for 20 minutes followed by incubation at 37° C. for the duration of the experiment).

C2984H cells transformed with pcI^{ts} ind⁺ modKlenTaq1 were used to test different modes of induction as shown in FIG. 2. A 500-ml baffle-bottomed Erlenmeyer flask containing 225 ml of TBS plus ampicillin was inoculated from an overnight culture of C2984K[pcI^{ts} ind⁺ modKlenTaq1] and incubated at 32° C. with shaking at 150 rpm. When the cell density reached 4 OD₆₀₀/ml, a Pre-induction Sample was removed and held on ice while the remainder of the culture was split into two subcultures, 100 ml each: 1) Chemical Induction Alone; and, 2) Temperature Induction Alone. In the case of the Chemical Induction Alone culture, nalidixic acid was added to a final concentration of 50 μg/ml and incubation was continued at 32° C. As a control, the Temperature Induction Alone culture was transferred to a 42° C. water bath, swirled for 20 minutes and then incubated at 37° C. with shaking for the duration of the experiment. This temperature induction regimen is used for lambda promoter-based expression plasmids under the control of a temperature sensitive lambda repressor. The cultures showed very similar growth curves. The nalidixic acid treated culture lagged behind the temperature induced culture. This may have been due to different incubation temperatures following induction. This may also be the result of induction of the SOS response by nalidixic acid. Nalidixic acid is a DNA gyrase inhibitor and the concentration used is sufficiently high to eventually inhibit chromosomal DNA replication.

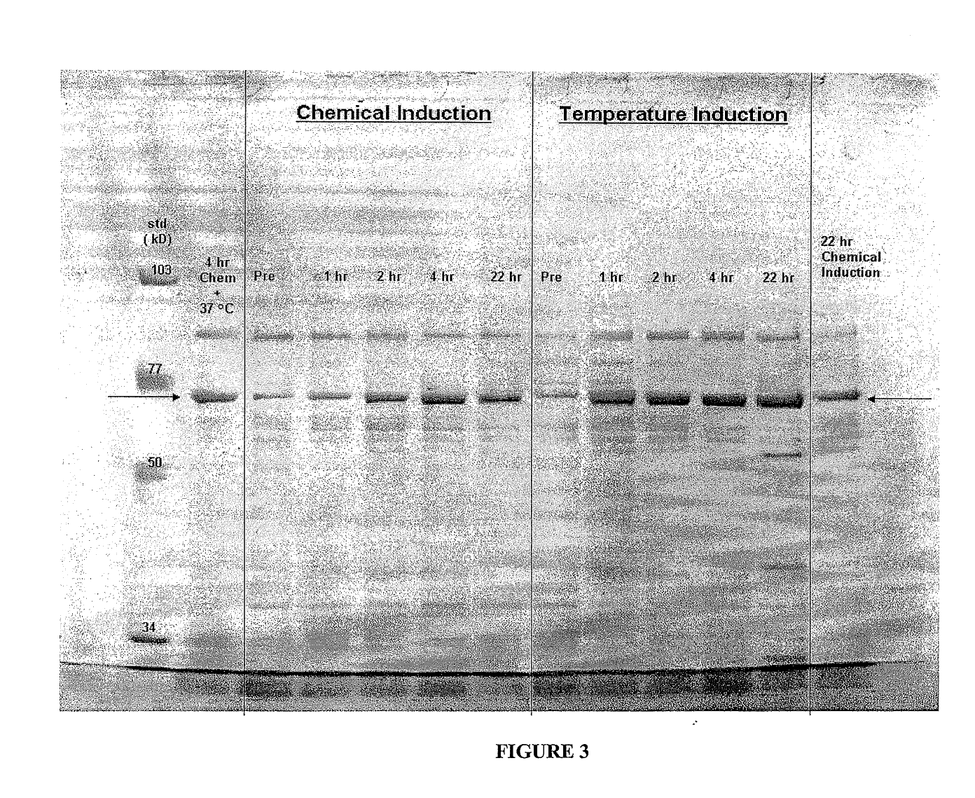

FIG. 3 depicts a comparison of protein yields for temperature-induced cells and chemically-induced cells in accordance with some embodiments of the present disclosure. Samples were removed from the cultures described in FIG. 2 at the times indicated (“Pre”: just prior to induction; 1, 2, 4, and 22 hours post induction) and processed as described above in Materials and Methods. Aliquots from the heat-treated samples equivalent to 0.1 OD₆₀₀ units of cells were analyzed by 8% SDS PAGE. Arrows indicate the expected migration position for modKlenTaq1, ˜64,000 Da.

Samples were removed at the times indicated and processed as described above in Materials and Methods for analysis by 8% SDS-PAGE as shown in FIG. 3. The gel shows only the heat treated samples for a comparison of the yields of modKlenTaq1. Each lane represents the protein from a cell sample equivalent to 0.1 OD₆₀₀ units. The banding patterns show that there was a low but detectable level of expression before induction. This may be due to partial inactivation of the repressor at 32° C. since subsequent experiments in which the cells were incubated at 30° C. showed no detectable expression in the pre-induction samples. Lambda expression systems generally have a single copy of the repressor as part of a pro-phage or cryptic lysogen. The results above indicate a higher concentration of repressor protein relative to other lambda expression systems even when the repressor gene was on-board the plasmid. This may be due to insufficient active repressor availability to fully inhibit transcription at 32° C.

The gel in FIG. 3 shows that the temperature-induction culture steadily accumulated modKlenTaq1 over the entire 26 hour time course of the experiment. Whereas, the chemically-induced culture showed slower accumulation with a maximum that occurred at 4 hours or at some time point between 4 and 26 hours since the 26 hour sample showed less staining than the 4 hour time point. Gels resolving the Total Cell Protein and Soluble Protein samples showed that modKlenTaq1 was only detected in the Total Cell Protein and Soluble Protein Samples and not lost to insoluble material (data not shown). Microscopic examination of the cells also indicated that the cells did not accumulate refractile bodies or become filamentous in either case following induction (data not shown). Since the repressor gene was present on the plasmid but there was only a single copy of the recA gene in the host chromosome, nalidixic acid induction alone may have been less efficient than temperature induction. Nevertheless, the 4 hour Chemical Induction Alone and the 4 hour Temperature Induction Alone samples are comparable.

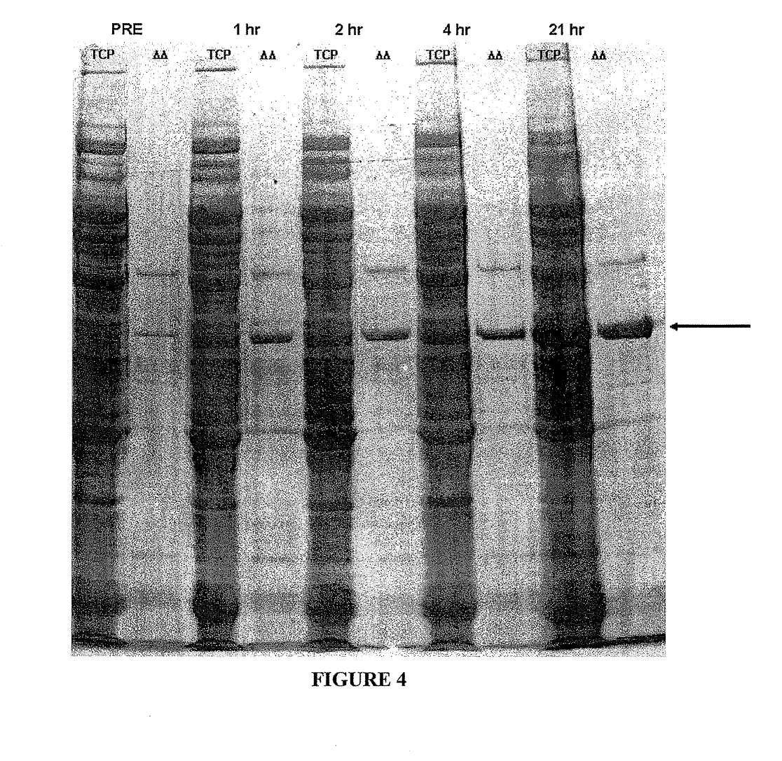

FIG. 4 depicts protein yields for cells that were induced by both chemical and temperature methods in accordance with some embodiments of the present disclosure. C2984H cells transformed with pcI^{ts} ind⁺ modKlenTaq1 were grown in 100 ml of TBS plus ampicillin in a 500-ml baffle-bottomed Erlenmeyer flask at 32° C. with shaking at 150 rpm. When the cells reached a density of 4 OD₆₀₀/ml the cultures were induced by adding nalidixic acid to a final concentration of 50 μg/ml as well as by increasing the incubator temperature to 37° C. Small shake-flasks under these conditions changed temperature from 32° C. to 37° C. Samples were removed at the times indicated and processed as described above in Materials and Methods and resolved on an 8% SDS-PAGE. Each lane represents the equivalent of 0.1 OD₆₀₀ of cells. FIG. 4 shows “TCP” (Total Cell Protein) and “ΔΔ” (Heat-treated Samples) for each of the time points. The arrow indicates the band for modKlenTaq.

FIG. 4 shows the effects to both adding nalidixic acid and simply increasing the incubator temperature dial to 37° C. The lanes represent the Total Cell Protein, “TCP,” and the Heat Treated Samples, “ΔΔ.” Following induction, the accumulation profile for modKlenTaq1 was comparable to that observed for the Temperature Alone experiments described above.

### Example 3

### Large Scale Shake Flask Cultures

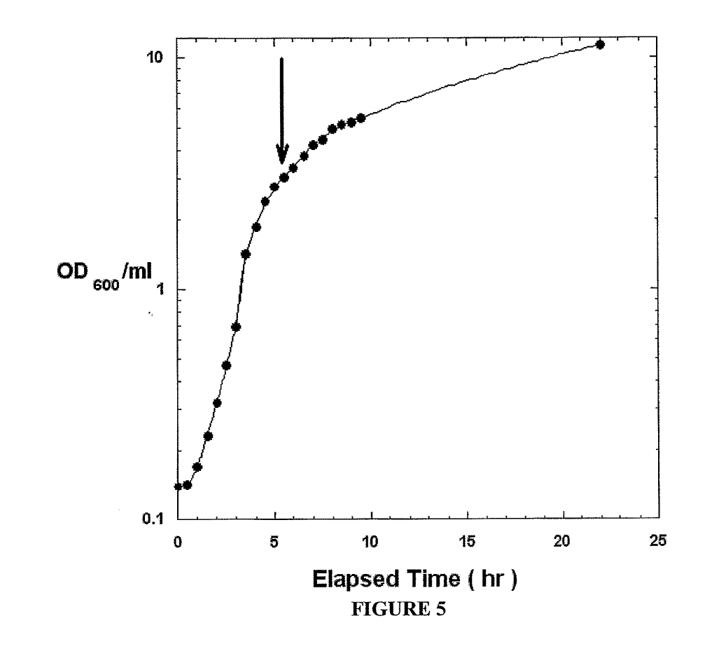

FIG. 5 shows a growth curve for large-scale shake-flask expression using chemical- and temperature-induction in accordance with some embodiments of the present disclosure. One of six 2.8-liter baffle-bottomed Fernbach flasks each containing 1.5 liters of TBS plus ampicillin (100 μg/ml) was monitored for cell growth. Pre-induction growth was at 30° C. with shaking at 125 rpm. At an OD₆₀₀/ml of 3, nalidixic acid was added to a final concentration of 50 μg/ml for chemical-induction and the temperature setting was increased to 37° C. for temperature-induction. The arrow indicates the time of induction. The final cell density was 11.2 OD₆₀₀ Units/ml; final cell wet weight was 96 gm.

FIG. 5 shows the growth curve for one of six identical 2.8-liter baffle-bottomed Fernbach flask cultures each containing 1.5 liters of TBS plus ampicillin and inoculated with C2984H cells carrying pcI^{ts} ind⁺ modKlenTaq1. The pre-induction incubation temperature was 30° C. to prevent pre-induction expression. One of the six flasks was used to monitor cell growth and to provide samples for gel analyses. The cells grew logarithmically up to a density of approximately 1.5 OD₆₀₀/ml with a doubling time of about 50 minutes. At cell densities above 1.5 OD₆₀₀/ml, in these large shake-flask cultures, the growth rate typically showed a steady decline. Smaller scale cultures using the same medium sustained logarithmic growth to a cell density above 8 OD₆₀₀/ml. This may be an effect cells being starved for oxygen rather than of the medium being depleted of an essential nutrient. When the cell density reached 3 OD₆₀₀/ml in the large shake-flasks (depicted by the arrow in FIG. 5), nalidixic acid was added to a final concentration of 50 μg/ml and the incubator temperature was increased to 37° C. The Lab-Line Model 3530-1 Orbital Shaker used in these experiments was able to increase the chamber temperature from 30° C. to 37° C. in 6 minutes. The temperature change within the flasks was much slower taking approximately 20 minutes. After 22 hours of incubation, the final cell density was 11.2 OD₆₀₀/ml and the final cell yield was 96 gm wet weight. All six flasks showed comparable growth.

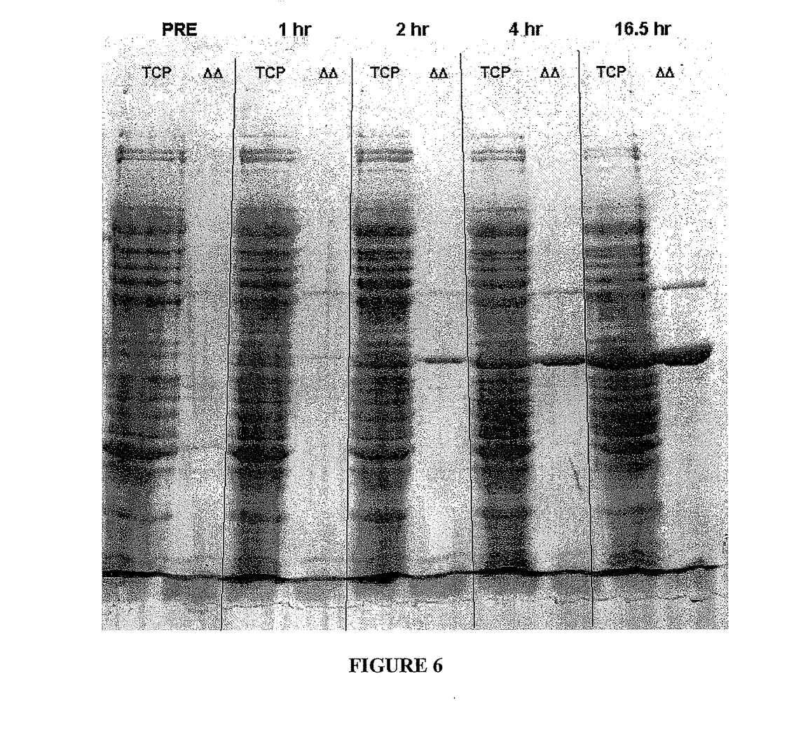

FIG. 6 depicts protein yields for large-scale shake-flask expression using temperature and chemical induction in accordance with some embodiments of the present disclosure. Samples were removed from the monitored flask described in FIG. 5 at the times indicated and processed as described above in Materials & Methods. Lanes 1-2: Pre-induction Total Cell Protein (TCP) and Heat-treated (ΔΔ); Lanes 3-4: 1 Hour TCP and ΔΔ; Lanes 5-6: 2 Hour TCP and ΔΔ; Lanes 7-8: 4 Hour TCP and ΔΔ; and, Lanes 9-10: 16.5 Hour TCP and ΔΔ. A sample equivalent 0.2 OD₆₀₀/ml was loaded onto each lane on an 8% gel as in shown FIG. 3. The arrow indicates modKlenTaq1 bands.

Samples were removed at the times indicated in FIG. 6 for gel analysis as described above. The gel shows Total Cell Protein and Heat-treated samples. Each lane was equivalent to 0.1 OD₆₀₀ units of cells. The gel shows no detectable accumulation of modKlenTaq1 in the pre-induction sample indicating more efficient control over transcription from the λP_{R} promoter at 30° C. Accumulation of modKlenTaq1 was much slower in the large flasks compared to the rate of accumulation observed for the smaller-scale cultures, however, the final yield after 22 hours of incubation was comparable in terms of cell-specific yield and final cell density.

### Example 4

### Purification of modKlenTaq1

Thermus aquaticus DNA polymerase 1 is known to be a remarkably thermostable enzyme. Its large fragment has been shown to be extremely thermostable. A two-step rapid purification protocol is disclosed, the protocol may be scaled-up. Frozen cell pellets were resuspended in Lysis Buffer and treated with lysozyme followed by sonication on ice to shear the DNA and reduce viscosity. The sonicate was slowly poured into an equal volume of Lysis Buffer in a water bath maintained at 80° C. forming a stirred slurry. The temperature of the slurry was never allowed to fall below 60° C. to ensure immediate denaturation of host proteins, especially proteases. Upon addition of the entire sonicate, the slurry was incubated with stirring at 80° C. for an additional 45 minutes. Following incubation, the slurry was cooled, the salt concentration was increased, and PEI was added drop wise to precipitate DNA. High salt prevented modKlenTaq1 from binding to the DNA in the PEI-precipitate. After centrifugation, the supernatant was loaded onto two tandem columns: a weak cation exchanger, BioRad-70; followed by an affinity column, Heparin-sepharose. The cation exchanger acted as a pre-column for the Heparin-sepharose column removing excess PEI. After washing both columns in tandem until the OD₂₈₀ returned to baseline, the affinity column was isolated.

FIG. 7 shows an elution profile of the purification of modKlenTaq1 in accordance with some embodiments of the present disclosure. A sample equivalent to 48 gm of cell wet weight was processed as described above in Materials and Methods and following centrifugation, the supernatant was pumped directly onto tandem BioRex-70 and Heparin-sepharose columns. After washing until the OD₂₈₀ signal returned to baseline, a 100 to 650 mM NaCl-gradient was used to elute only the Heparin-sepharose column. ModKlenTaq1 eluted from the column at approximately 400 mM. Each column fraction was 14 ml. ModKlenTaq1 was eluted from the Heparin-sepharose column using a 5.5 CV linear gradient (100 mM to 650 mM NaCl) as shown in FIG. 7.

FIG. 8 depicts gel analysis of the column fractions. Five μL aliquots from peak column fractions were analyzed by 12% SDS-PAGE. The arrow indicates the modKlenTaq1 band. The major peak was modKlenTaq1 as shown by gel analysis in FIG. 8. The final yield of purified modKlenTaq1 was 285 mg.

modKlenTaq1 Expression Using Chemical vs. Temperature Induction

The lambda rightward promoter, λP_{R}, is normally active during the lytic cycle of this temperate bacteriophage and is repressed during lysogeny. Efficient repression is necessary to maintain the lysogenic state and is provided by binding of the lambda repressor, λcI, to the λO_{R} operator which, in turn represses the so-called anti-terminator gene, λcro. As long as the repressor concentration is moderately high, λcro remains repressed. Therefore, the region of the lamdba genome spanning the λcI repressor, λO_{R} and λP_{R} sequences is of special interest as a self-contained transcriptional control unit. The wild-type λcI repressor may be inactivated through self-proteolysis via a host encoded, activated RecA protein that acts as a co-protease. Treatment of E. coli with mitomycin-C or nalidixic acid induces recA expression and has been used to induce phage production from lysogens and to induce heterologous gene expression on plasmid constructs. For example, the leftward promoter has been used to overexpress the gene encoding transcription factor rho to very high levels using nalidixic acid for chemical-induced in recA⁺ host cells that were also lambda cI⁺ cryptic lysogens. Taq DNA polymerase has been expressed at 1-2% of the total cellular protein using a pPR-TGATG-1 expression vector with the temperature sensitive lambda repressor, λcI857, onboard the plasmid. Most expression vectors utilizing either of the lambda promoters, λP_{L} or λP_{R} or both, have been controlled by the temperature sensitive λcI857 repressor and unless the repressor is on-board the plasmid are limited to lysogenic hosts. The λcI857 repressor carries two mutations, temperature sensitivity (A67T) and ind 1 (E118K) or resistance to RecA protein cleavage. An expression system that relies on the λcI857 repressor may be induced using temperature.

Raising the temperature of several flasks rapidly has been a problem using shake-flask cultures. The teachings of the present disclosure, in some embodiments, provide a novel expression construct that comprises a lambda repressor gene, λcI^{ts} ind⁺, that provides for temperature and/or chemical induction. As shown in FIG. 1, the expression vector, pcI^{ts} ind⁺, comprises a region from lambda, λcI857 ind 1 Sam7, that includes the λcI857 ind 1 repressor, the λP_{R} promoter and the start codon of the λcro gene. In some embodiments, the repressor may be back-mutated to be ind⁺ while maintaining the temperature sensitive phenotype. Restoring ind⁺ may remove a Hind3 restriction site (T to C at λ37589) thereby enabling a method to identify back-mutation clones. In some embodiments, the coding region for the λcro gene may be deleted and a unique Nde1 insertion restriction site constructed to overlap its ATG initiation codon. This construction may add an additional base and change a base in the sequence between the Shine-Dalgarno site and the initiator codon ( . . . AGGAGGTTGT-ATG . . . to . . . AGGAGGTTcaT-ATG . . . ).

Despite the high percentage of GC content of the coding sequence for modKlenTaq1, it may not be necessary to use a “stutter-stop-start” pre-coding segment to avoid secondary structure in the mRNA. In some embodiments, the coding sequence for modKlenTaq1 may be linked directly to the ATG start codon at the Nde1 site described above. In some embodiments, a unique Sph1 3′-insertion restriction site may be constructed immediately ahead of the T1T2 ribosomal terminators from the E. coli rrnB operon in the plasmid pUC19-T1T2. This plasmid has as its origin of replication the high copy number pUC ori. In some embodiments, a portion of the Taq DNA polymerase 1 gene may be amplified using PCR primers containing the same cryptic restriction sites to allow insertion of the modKlenTaq 1 coding region into the Nde1 and Sph1 sites as shown in FIG. 1 generating the plasmid, pcI^{ts} ind⁺ modKlenTaq1. This version of the Taq DNAP1 gene encodes the C-terminal amino acids 281-832 plus 7 additional amino acids added at its N-terminal end for improved solubility, MGKRKST.

In some embodiments, the expression plasmid, pcI^{ts} ind⁺ modKlenTaq1, may be transformed into C2984H cells (recA⁺). recA⁺ hosts may be far more robust than recA⁻ hosts that may be used for expression of recombinant enzymes. C2984H grown at 30° C. showed doubling times as short as recA⁻ strains like DH5α cells grown at 37° C.

For example, small volume cultures were used to survey the effects of temperature- vs. chemical-induction. FIG. 2 shows that the growth curves for either type of induction were similar. FIG. 3 shows a gel for the heat-treated samples removed at the various times as indicated from each culture. In initial experiments, the pre-induction incubation temperature was 32° C. and a low level of expression was observed in the pre-induction samples. All large scale experiments described herein were conducted at a pre-induction temperature of 30° C. and no pre-induction expression was detected. FIG. 3 shows that both induction schemes were successful in expressing modKlenTaq1. In some embodiments, temperature-induction alone was more efficient than chemical-induction alone with respect to the accumulation rate and final overall specific cell yield of modKlenTaq1 as observed from the about 2 to 3-fold darker staining bands for all samples taken from the temperature-induced culture. A temperature shift may inactivate all repressor molecules at the time of induction. The presence of a single copy of the recA gene in the host chromosome relative to the lambda repressor present on a high copy number plasmid, may result in low level of expression of RecA as compared to the level of repressor molecules in the cell. In some embodiments, continued incubation at lower temperatures following the addition of nalidixic acid may allow continued expression of active repressor. In some embodiments, chemical-induction induced modKlenTaq1 to high specific cell yields and the 4 hour time points were comparable.

In some embodiments, combined induction may be more efficient as accumulation of modKlenTaq1 in chemically-induced cultures lagged behind the rate observed for temperature-induced cultures (where levels of RecA protein were overwhelmed by repressor concentrations and by continued synthesis of active repressor). FIG. 4 shows the Total Cell Protein and Heat-treated Protein samples for a small scale culture that was induced by the addition of nalidixic acid and increasing the incubator temperature to 37° C. The accumulation and final specific cell yield of modKlenTaq1 were comparable to the results shown in FIG. 3 for the Temperature Induction Alone culture. Increased temperature (37° C. following the addition of nalidixic acid) reduces the number of active repressor molecules that were cleaved by RecA protein. In some embodiments, the disclosure provides a scaled-up method for producing larger quantities of the protein using the expression vector of the disclosure comprising a) addition of nalidixic acid; and b) raising the incubator temperature, suing more than one shake-flasks with larger volumes. In some embodiments, the method may involve a “temperature-jump” to 42° C. In some embodiments, the scaled-up method for production is easier to perform than the temperature jump method.

### Example 5

### Large-Scale Shake-Flask Expression Using Both Chemical and Temperature Induction

FIG. 5 shows a growth curve for one of 6 flasks (each 1.5 liters of TB with Salts and Ampicillin). The pre-induction incubation temperature was 30° C. The cells showed a doubling time of approximately 50 minutes during log phase growth up to a density of about 2 OD₆₀₀/m. Unlike the small volume cultures, the larger volume flasks showed decreasing growth rates above a cell density of 2 A₆₀₀/ml. Since the smaller volume cultures were able to sustain logarithmic growth to a cell density above 8 A₆₀₀/ml as shown in FIG. 2, the decreasing growth rate may be due to the larger volume flasks being less efficient at air exchange rather than the cultures being depleted of an essential nutrient. As the growth rate showed a steady decline at cell densities above 2 OD₆₀₀/ml, induction was performed earlier. At a cell density of 3 OD₆₀₀/ml, nalidixic acid was added to a final concentration of 50 μg/ml and the temperature controller on the shaker incubator was raised to 37° C. Samples were removed and processed as described at the times indicated in FIG. 6. The final cell density after 22 hours of growth (16.5 hours elapsed time from the time of induction) reached 11.2 A₆₀₀/ml yielding 96 gm total cell wet weight or 10.6 gm/liter. Samples were processed for Total Cell Protein and Heat Treated Supernatant. ModKlenTaq1 was not detectable before induction. Post induction, modKlenTaq1 appeared at 2 hours and steadily increased for the duration of the experiment as shown in both the Total Cell Protein and Heat Treated fractions.

### Example 6

### Purification of modKlenTaq1

Taq DNA polymerase is a thermostable enzyme and has been shown to have a half-life in excess of 60 minutes at 95° C. The present disclosure provides a rapid two-step purification protocol including a heat-treatment step plus affinity chromatography to purify modKlenTaq1. The cell lysate was incubated at 80° C. for 45 minutes to precipitate most E. coli proteins. DNA was removed by precipitation with polyethyleneimine and the resulting supernatant after pelleting cell debris and denatured proteins was pumped directly onto two columns in tandem: the first column was a weak-cation exchanger to remove excess polyethyleneimine (BioRex-70) and the second column was an affinity column, Heparin-sepharose. ModKlenTaq1 bound tightly to the affinity column, eluting at 0.4 M NaCl as the major peak with a small shoulder representing a faster migrating species on SDS-PAGE. The final total yield of purified modKlenTaq1 was 285 mg from 9 liters of culture in 6 flasks or 31.6 mg/L or 3 mg/gm cell wet weight.

One example of a plasmid sequence as described above is as follows:

| (SEQ ID NO: 1) | |
|---|---|
| 1 | CATATGGGTA AACGTAAATC TACTGCCTTT CTGGAGAGGC TTGAGTTTGG |
| 51 | CAGCCTCCTC CACGAGTTCG GCCTTCTGGA AAGCCCCAAG GCCCTGGAGG |
| 101 | AGGCCCCCTG GCCCCCGCCG GAAGGGGCCT TCGTGGGCTT TGTGCTTTCC |
| 151 | CGCAAGGAGC CCATGTGGGC CGATCTTCTG GCCCTGGCCG CCGCCAGGGG |
| 201 | GGGCCGGGTC CACCGGGCCC CCGAGCCTTA TAAAGCCCTC AGGGACCTGA |
| 251 | AGGAGGCGCG GGGGCTTCTC GCCAAAGACC TGAGCGTTCT GGCCCTGAGG |
| 301 | GAAGGCCTTG GCCTCCCGCC CGGCGACGAC CCCATGCTCC TCGCCTACCT |
| 351 | CCTGGACCCT TCCAACACCA CCCCCGAGGG GGTGGCCCGG CGCTACGGCG |
| 401 | GGGAGTGGAC GGAGGAGGCG GGGGAGCGGG CCGCCCTTTC CGAGAGGCTC |
| 451 | TTCGCCAACC TGTGGGGGAG GCTTGAGGGG GAGGAGAGGC TCCTTTGGCT |
| 501 | TTACCGGGAG GTGGAGAGGC CCCTTTCCGC TGTCCTGGCC CACATGGAGG |
| 551 | CCACGGGGGT GCGCCTGGAC GTGGCCTATC TCAGGGCCTT GTCCCTGGAG |
| 601 | GTGGCCGAGG AGATCGCCCG CCTCGAGGCC GAGGTCTTCC GCCTGGCCGG |
| 651 | CCACCCCTTC AACCTCAACT CCCGGGACCA GCTGGAAAGG GTCCTCTTTG |
| 701 | ACGAGCTAGG GCTTCCCGCC ATCGGCAAGA CGGAGAAGAC CGGCAAGCGC |
| 751 | TCCACCAGCG CCGCCGTCCT GGAGGCCCTC CGCGAGGCCC ACCCCATCGT |
| 801 | GGAGAAGATC CTGCAGTACC GGGAGCTCAC CAAGCTGAAG AGCACCTACA |
| 851 | TTGACCCCTT GCCGGACCTC ATCCACCCCA GGACGGGCCG CCTCCACACC |
| 901 | CGCTTCAACC AGACGGCCAC GGCCACGGGC AGGCTAAGTA GCTCCGATCC |
| 951 | CAACCTCCAG AACATCCCCG TCCGCACCCC GCTTGGGCAG AGGATCCGCC |
| 1001 | GGGCCTTCAT CGCCGAGGAG GGGTGGCTAT TGGTGGCCCT GGACTATAGC |
| 1051 | CAGATAGAGC TCAGGGTGCT GGCCCACCTC TCCGGCGACG AGAACCTGAT |
| 1101 | CCGGGTCTTC CAGGAGGGGC GGGACATCCA CACGGAGACC GCCAGCTGGA |
| 1151 | TGTTCGGCGT CCCCCGGGAG GCCGTGGACC CCCTGATGCG CCGGGCGGCC |
| 1201 | AAGACCATCA ACTTCGGGGT CCTCTACGGC ATGTCGGCCC ACCGCCTCTC |
| 1251 | CCAGGAGCTA GCCATCCCTT ACGAGGAGGC CCAGGCCTTC ATTGAGCGCT |
| 1301 | ACTTTCAGAG CTTCCCCAAG GTGCGGGCCT GGATTGAGAA GACCCTGGAG |
| 1351 | GAGGGCAGGA GGCGGGGGTA CGTGGAGACC CTCTTCGGCC GCCGCCGCTA |
| 1401 | CGTGCCAGAC CTAGAGGCCC GGGTGAAGAG CGTGCGGGAG GCGGCCGAGC |
| 1451 | GCATGGCCTT CAACATGCCC GTCCAGGGCA CCGCCGCCGA CCTCATGAAG |
| 1501 | CTGGCTATGG TGAAGCTCTT CCCCAGGCTG GAGGAAATGG GGGCCAGGAT |
| 1551 | GCTCCTTCAG GTCCACGACG AGCTGGTCCT CGAGGCCCCA AAAGAGAGGG |
| 1601 | CGGAGGCCGT GGCCCGGCTG GCCAAGGAGG TCATGGAGGG GGTGTATCCC |
| 1651 | CTGGCCGTGC CCCTGGAGGT GGAGGTGGGG ATAGGGGAGG ACTGGCTCTC |
| 1701 | CGCCAAGGAG TGAGCATGCA GTAGGGAACT GCCAGGCATC AAATAAAACG |
| 1751 | AAAGGCTCAG TCGAAAGACT GGGCCTTTCG TTTTATCTGT TGTTTGTCGG |
| 1801 | TGAACGCTCT CCTGAGTAGG ACAAATCCGC CGGGAGCGGA TTTGAACGTT |
| 1851 | GCGAAGCAAC GGCCCGGAGG GTGGCGGGCA GGACGCCCGC CATAAACTGC |
| 1901 | CAGGCATCAA ATTAAGCAGA AGGCCATCCT GACGGATGGC CTTTTTGCGT |
| 1951 | TTCTACAAAC TCTTTTTGTT TATTTTTCTA AATACATTCA AATATGTATC |
| 2001 | CGCTCATGAG ACAATAGATC TAAGCTTGGC GTAATCATGG TCATAGCTGT |
| 2051 | TTCCTGTGTG AAATTGTTAT CCGCTCACAA TTCCACACAA CATACGAGCC |
| 2101 | GGAAGCATAA AGTGTAAAGC CTGGGGTGCC TAATGAGTGA GCTAACTCAC |
| 2151 | ATTAATTGCG TTGCGCTCAC TGCCCGCTTT CCAGTCGGGA AACCTGTCGT |
| 2201 | GCCAGCTGCA TTAATGAATC GGCCAACGCG CGGGGAGAGG CGGTTTGCGT |
| 2251 | ATTGGGCGCT CTTCCGCTTC CTCGCTCACT GACTCGCTGC GCTCGGTCGT |
| 2301 | TCGGCTGCGG CGAGCGGTAT CAGCTCACTC AAAGGCGGTA ATACGGTTAT |
| 2351 | CCACAGAATC AGGGGATAAC GCAGGAAAGA ACATGTGAGC AAAAGGCCAG |
| 2401 | CAAAAGGCCA GGAACCGTAA AAAGGCCGCG TTGCTGGCGT TTTTCCATAG |
| 2451 | GCTCCGCCCC CCTGACGAGC ATCACAAAAA TCGACGCTCA AGTCAGAGGT |
| 2501 | GGCGAAACCC GACAGGACTA TAAAGATACC AGGCGTTTCC CCCTGGAAGC |
| 2551 | TCCCTCGTGC GCTCTCCTGT TCCGACCCTG CCGCTTACCG GATACCTGTC |
| 2601 | CGCCTTTCTC CCTTCGGGAA GCGTGGCGCT TTCTCATAGC TCACGCTGTA |
| 2651 | GGTATCTCAG TTCGGTGTAG GTCGTTCGCT CCAAGCTGGG CTGTGTGCAC |
| 2701 | GAACCCCCCG TTCAGCCCGA CCGCTGCGCC TTATCCGGTA ACTATCGTCT |
| 2751 | TGAGTCCAAC CCGGTAAGAC ACGACTTATC GCCACTGGCA GCAGCCACTG |
| 2801 | GTAACAGGAT TAGCAGAGCG AGGTATGTAG GCGGTGCTAC AGAGTTCTTG |
| 2851 | AAGTGGTGGC CTAACTACGG CTACACTAGA AGAACAGTAT TTGGTATCTG |
| 2901 | CGCTCTGCTG AAGCCAGTTA CCTTCGGAAA AAGAGTTGGT AGCTCTTGAT |
| 2951 | CCGGCAAACA AACCACCGCT GGTAGCGGTG GTTTTTTTGT TTGCAAGCAG |
| 3001 | CAGATTACGC GCAGAAAAAA AGGATCTCAA GAAGATCCTT TGATCTTTTC |
| 3051 | TACGGGGTCT GACGCTCAGT GGAACGAAAA CTCACGTTAA GGGATTTTGG |
| 3101 | TCATGAGATT ATCAAAAAGG ATCTTCACCT AGATCCTTTT AAATTAAAAA |
| 3151 | TGAAGTTTTA AATCAATCTA AAGTATATAT GAGTAAACTT GGTCTGACAG |
| 3201 | TTACCAATGC TTAATCAGTG AGGCACCTAT CTCAGCGATC TGTCTATTTC |
| 3251 | GTTCATCCAT AGTTGCCTGA CTCCCCGTCG TGTAGATAAC TACGATACGG |
| 3301 | GAGGGCTTAC CATCTGGCCC CAGTGCTGCA ATGATACCGC GAGACCCACG |
| 3351 | CTCACCGGCT CCAGATTTAT CAGCAATAAA CCAGCCAGCC GGAAGGGCCG |
| 3401 | AGCGCAGAAG TGGTCCTGCA ACTTTATCCG CCTCCATCCA GTCTATTAAT |
| 3451 | TGTTGCCGGG AAGCTAGAGT AAGTAGTTCG CCAGTTAATA GTTTGCGCAA |
| 3501 | CGTTGTTGCC ATTGCTACAG GCATCGTGGT GTCACGCTCG TCGTTTGGTA |
| 3551 | TGGCTTCATT CAGCTCCGGT TCCCAACGAT CAAGGCGAGT TACATGATCC |
| 3601 | CCCATGTTGT GCAAAAAAGC GGTTAGCTCC TTCGGTCCTC CGATCGTTGT |
| 3651 | CAGAAGTAAG TTGGCCGCAG TGTTATCACT CATGGTTATG GCAGCACTGC |
| 3701 | ATAATTCTCT TACTGTCATG CCATCCGTAA GATGCTTTTC TGTGACTGGT |
| 3751 | GAGTACTCAA CCAAGTCATT CTGAGAATAG TGTATGCGGC GACCGAGTTG |
| 3801 | CTCTTGCCCG GCGTCAACAC GGGATAATAC CGCGCCACAT AGCAGAACTT |
| 3851 | TAAAAGTGCT CATCATTGGA AAACGTTCTT CGGGGCGAAA ACTCTCAAGG |
| 3901 | ATCTTACCGC TGTTGAGATC CAGTTCGATG TAACCCACTC GTGCACCCAA |
| 3951 | CTGATCTTCA GCATCTTTTA CTTTCACCAG CGTTTCTGGG TGAGCAAAAA |
| 4001 | CAGGAAGGCA AAATGCCGCA AAAAAGGGAA TAAGGGCGAC ACGGAAATGT |
| 4051 | TGAATACTCA TACTCTTCCT TTTTCAATAT TATGTAAGCA GACAGTTTTA |
| 4101 | TTGTTCATGA TGATATATTT TTATCTTGTG CAATGTAACA TCAGAGATTT |
| 4151 | TGAGACACAA CGTGGCTTTG TTGAATAAAT CGAACTTTTG CTGAGTTGAC |
| 4201 | TCCCCGCGCG GACATTAATT GCGTTGCGCT CACTGCCCGC TTTCCAGTCG |
| 4251 | GGAAACCTGT CGTGCCAGCT GCATTAATGA ATCGGCCAAC GCGCGGGGAG |
| 4301 | AGGCGGTTTG CGTATTGGGC GCCATAGACG TCTTTGAATT GTTATCAGCT |
| 4351 | ATGCGCCGAC CAGAACACCT TGCCGATCAG CCAAACGTCT CTTCAGGCCA |
| 4401 | CTGACTAGCG ATAACTTTCC CCACAACGGA ACAACTCTCA TTGCATGGGA |
| 4451 | TCATTGGGTA CTGTGGGTTT AGTGGTTGTA AAAACACCTG ACCGCTATCC |
| 4501 | CTGATCAGTT TCTTGAAGGT AAACTCATCA CCCCCAAGTC TGGCTATGCA |
| 4551 | GAAATCACCT GGCTCAACAG CCTGCTCAGG GTCAACGAGA ATTAACATTC |
| 4601 | CGTCAGGAAA GCTTGGCTTG GAGCCTGTTG GTGCGGTCAT GGAATTACCT |
| 4651 | TCAACCTCAA GCCAGAATGC AGAATCACTG GCTTTTTTGG TTGTGCTTAC |
| 4701 | CCATCTCTCC GCATCACCTT TGGTAAAGGT TCTAAGCTCA GGTGAGAACA |
| 4751 | TCCCTGCCTG AACATGAGAA AAAACAGGGT ACTCATACTC ACTTCTAAGT |
| 4801 | GACGGCTGCA TACTAACCGC TTCATACATC TCGTAGATTT CTCTGGCGAT |
| 4851 | TGAAGGGCTA AATTCTTCAA CGCTAACTTT GAGAATTTTT GTAAGCAATG |
| 4901 | CGGCGTTATA AGCATTTAAT GCATTGATGC CATTAAATAA AGCACCAACG |
| 4951 | CCTGACTGCC CCATCCCCAT CTTGTCTGCG ACAGATTCCT GGGATAAGCC |
| 5001 | AAGTTCATTT TTCTTTTTTT CATAAATTGC TTTAAGGCGA CGTGCGTCCT |
| 5051 | CAAGCTGCTC TTGTGTTAAT GGTTTCTTTT TTGTGCTCAT ACGTTAAATC |
| 5101 | TATCACCGCA AGGGATAAAT ATCTAACACC GTGCGTGTTG ACTATTTTAC |
| 5151 | CTCTGGCGGT GATAATGGTT GCATGTACTA AGGAGGTT |

### Example 7

### Construction of LdK39 Vector

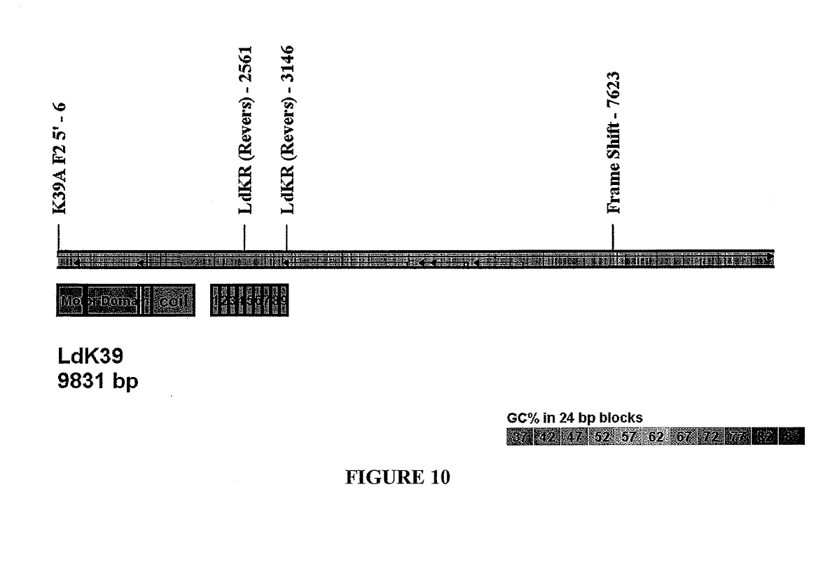

FIG. 10 depicts a partial restriction map of the LdK39 gene. A nucleic acid containing the LdK39 gene was cut with the restriction enzymes Nde1 and Sph1 to yield a fragment. This fragment was subcloned into pUC19. This formed a base plasmid from which a final expression vector was prepared.

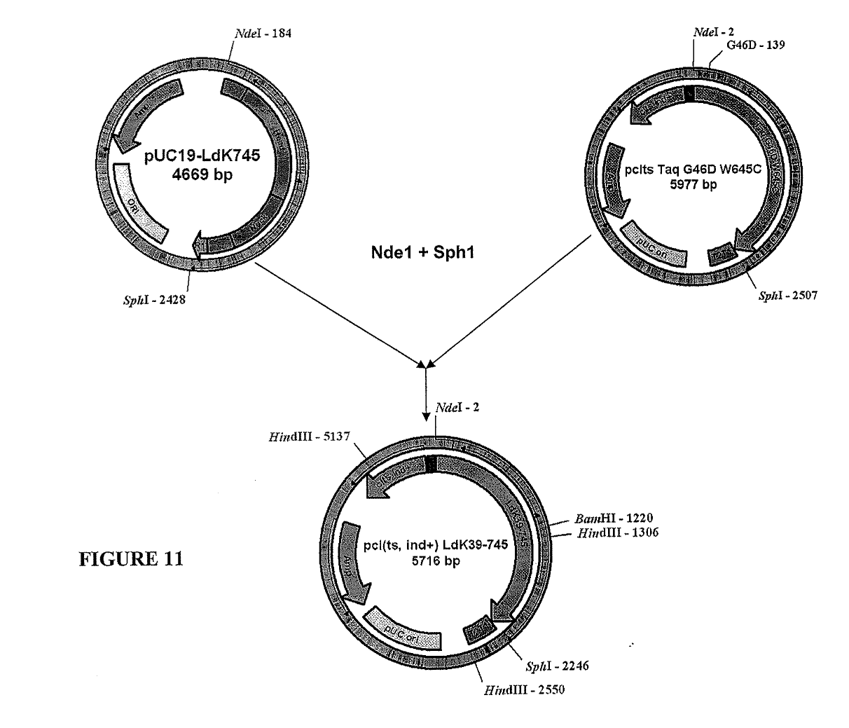

The final expression vector was prepared as shown in FIG. 11. The pUC19 vector containing the LdK39 gene fragment was cut with Nde1 and Sph1 to free the LdK39 fragment. This fragment was then subcloned into Nde1 and Sph1 cut fragment of the pcl^{ts} Taq G46D W645C vector. The resulting final vector contained an LdK39 fragment able to code a 745 amino acid protein in a pcl^{ts} ind⁺ vector.

### Example 8

### Expression Testing

C2984H cells were transformed with the pcl^{ts} ind⁺ LdK39-745 vector of Example 7. A 500-ml baffle-bottomed Erlenmeyer flask containing 125 mL of TBS plus ampicillin was inoculated from an overnight culture of C2984H[pcl^{ts} ind⁺ LdK39-745] and incubated at 30° C. with shaking at 150 rmp. When cell density reached 4 OD₆₀₀/mL, a Pre-induction sample was removed and held on ice while the remainder of the culture was split into two subcultures, 60 mL each: 1) Chemical Induction Alone; and 2) Temperature and Chemical Induction. In the case of both samples, nalidixic acid was added to a final concentration of 50 μg/mL. For the Chemical Induction Alone sample, incubation was continued at 30° C. For the Temperature and Chemical Induction sample, the culture was transferred to a 42° C. water bath, swirled for 20 minutes, and then incubated at 37° C. with shaking for the duration of the experiment. Samples were taken from both cultures 1, 2, 4 and 26 hours post-induction

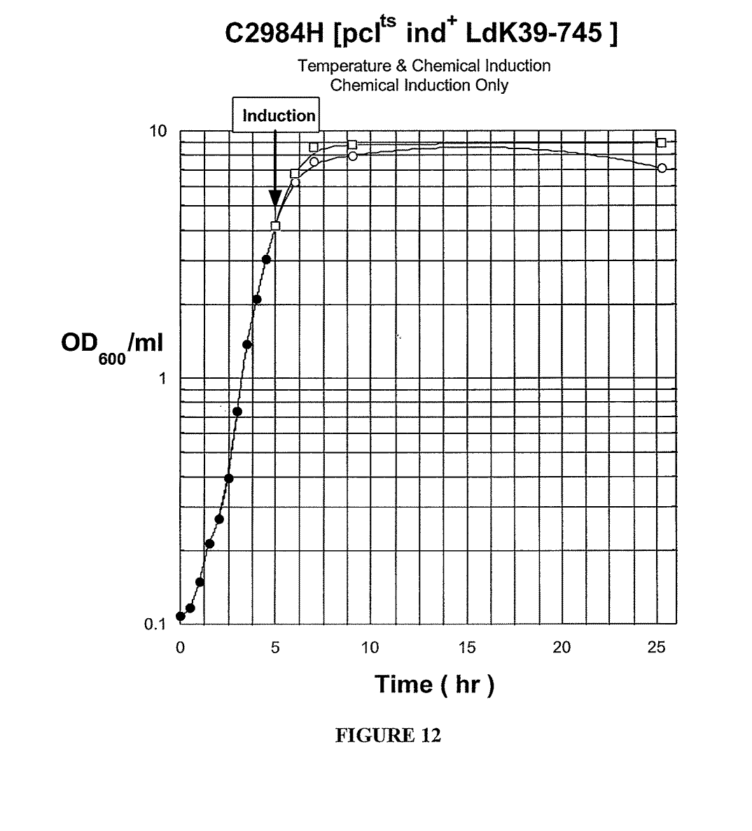

FIG. 12 shows growth curves for these samples. The final OD/mL for the Chemical Induction Only sample was 7. The final OD/mL for the Temperature and Chemical Induction sample was 8.9.

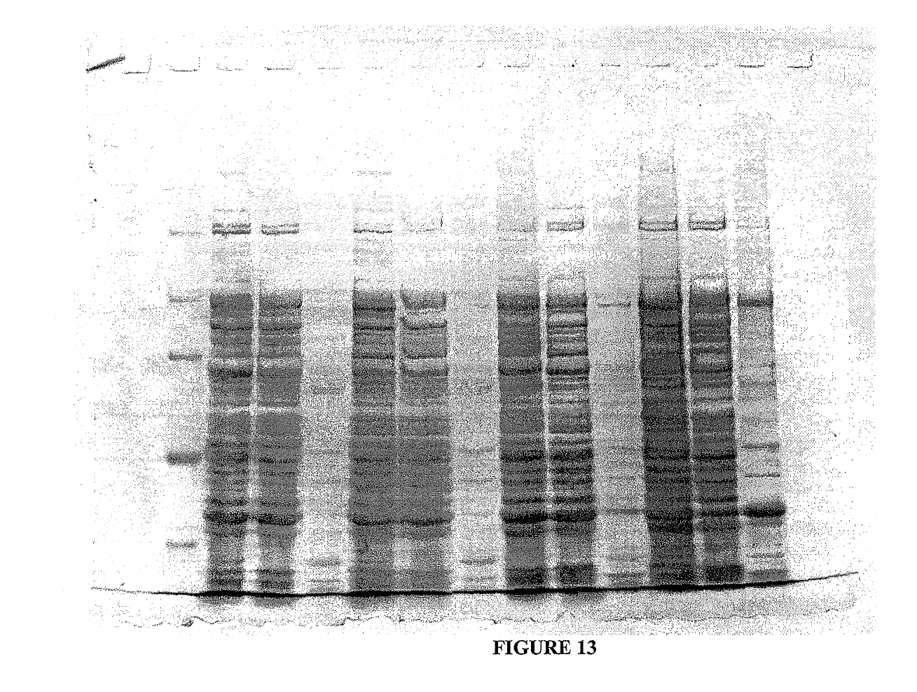

FIG. 13 depicts a comparison of protein yields for the two samples at the times tested. Samples were processed as described in Example 1. Aliquots from each sample equivalent to 0.1 OD₆₀₀ units of cells were analyzed by 8% SDS PAGE. Arrows indicate the expected migration position for the 745 amino acid LdK39 protein.

The pcl^{ts} ind⁺ LdK39-745 vector was modified to add a Flag-tag to the LdK39 protein. C2984H cells were transformed with this modified vector and grown as described previously in this example. The cells were subject to both chemical and temperature induction. Cell protein was extracted as described in the “Gel Samples” portion of Example 1. Samples representing total cell protein, soluble protein, and insoluble protein were prepared. The samples were also eluted through an affinity column as described in Example 1. Both the cell protein and affinity column samples were used to prepare a Western blot that was then probed with an anti-Flag antibody (Sigma, St. Louis, Mo.). Flag-tagged LdK745 was clearly identified in the samples that had been induced and was absent in the pre-induction samples.

Thus, the pcl^{ts} ind⁺ LdK39-745 vector or similar vectors containing LdK fragments may be used for high-yield production of LdK protein or protein fragments. These LdK proteins or protein fragments may be immunogenic and may be useful in inducing a protective immune response.

As will be understood by those skilled in the art, other equivalent or alternative methods, devices, systems and compositions for generating workable amounts of enzymes according to embodiments of the present disclosure may be envisioned without departing from the essential characteristics thereof. For example, where a range is disclosed, the end points may be regarded as guides rather than strict limits. In some embodiments, methods, compositions, devices, and/or systems may be adapted to accommodate ergonomic interests, aesthetic interests, scale, or any other interests. Such modifications may influence other steps, structures and/or functions (e.g., positively, negatively, or insubstantially). A negative influence on function may include, for example, a loss of fractionation capacity and/or resolution. Yet, this loss may be deemed acceptable, for example, in view of offsetting ergonomic, aesthetic, scale, cost, or other factors.

In some embodiments, a device of the disclosure may be manufactured in either a handheld or a tabletop configuration, and may be operated sporadically, intermittently, and/or continuously. Individuals skilled in the art would recognize that additional separation methods may be incorporated, e.g., to partially or completely remove proteins, lipids, carbohydrates, nucleic acids, salts, solvents, detergents, and/or other materials from a test sample. Also, the temperature (e.g. incubation temperature or induction temperature), pressure, and acceleration at which each step is performed may be varied.

All or part of a system of the disclosure may be configured to be disposable and/or reusable. From time to time, it may be desirable to clean, repair, and/or refurbish at least a portion of a device and/or system of the disclosure. For example, a reusable component may be cleaned to inactivate, remove, and/or destroy one or more contaminants. Individuals skilled in the art would recognize that a cleaned, repaired, and/or refurbished component is within the scope of the disclosure.

These equivalents and alternatives along with obvious changes and modifications are intended to be included within the scope of the present disclosure. Moreover, one of ordinary skill in the art will appreciate that no embodiment, use, and/or advantage is intended to universally control or exclude other embodiments, uses, and/or advantages. Expressions of certainty (e.g., “will,” “are,” and “can not”) may refer to one or a few example embodiments without necessarily referring to all embodiments of the disclosure. Accordingly, the foregoing disclosure is intended to be illustrative, but not limiting, of the scope of the disclosure.

### REFERENCES

The following references, to the extent that they provide exemplary procedural or other details supplementary to those set forth herein, are specifically incorporated herein, in their entirety, by reference:

A. Villaverde, A. Benito, E. Viaplana, R. Cubarsi. Fine regulation of cI857-controlled gene expression in continuous culture of recombinant Escherichia coli by temperature. Appl. Environ. Microbiol. 59 (1993) 3485-3487.

A. Dey, P. Sharma, N. S. Redhu, S. Singh. Kinesin Motor Domain of Leishmania Donovani as future vaccine candidate. Clin. Vaccine Immunology, online pre-publication, Mar. 19, 2008.

C. Yanish-Perron, J. Vieira, J. Messing. Improved M13 phage and host strains: nucleotide sequences of the M13mp18 and pUC19 vectors. Gene 33 (1985) 103-119.

D. R. Engleke, A. Krikos, M. E. Bruck, D. Ginsburg. Purification of Thermus aquaticus DNA polymerase expressed in E. coli. Analyt. Biochem. 191 (1990): 396-400.

E. Remaut, P. Stanssens, W. Fiers. Plasmid vectors for high-efficiency expression controlled by the PL promoter of coliphage lambda. Gene 15 (1981) 81-93.

F. Baneyx. Recombinant protein expression in Escherichia coli. Curr. Opin. Biotechnol. 10 (1999) 411-421.

J. Brosius, A. Ulrich, M. A. Baker, A. Gray, T. J. Dull, R. G. Gutell, H. F. Noller. Construction and fine mapping of recombinant plasmids containing the rrnB ribosomal RNA operon of E. coli. Plasmid 6 (1981) 112-118.

J. A. Mustard, J. W. Little. Analysis of Escherichia coli recA interactions with lexA. λcI, and ummD by site-directed mutagenesis of recA. J. Bacteriol. 182 (2000) 1659-1670.

J. E. Mott, R. A. Grant, Y.-S. Ho, T. Platt. Maximizing gene expression from plasmid vectors containing the λP_{L} promoter: Strategies for over producing transcription terminator factor ρ. Proc. Natl. Acad. Sci. USA 82 (1985) 88-92.

J. H. Miller. Experiments in Molecular Genetics. (1972) Cold Spring Harbor Laboratory Press, NY.

J. W. Roberts and R. Devoret (1983) in Lambda II, Hendrix, R., Roberts, J., Stahl, F., and Weisberg, R., eds. Cold Spring Harbor Laboratory Press, Cold Spring Harbor, N.Y., pp. 130-133.

K. A. Johnson. Rapid quench kinetic analysis of polymerases, adenosinetriphosphatases, and enzyme intermediates. Methods in Enzymol. 249 (1995) 38-61.

K. D. Tartoff, C. A. Hobbs. Improved media for growing plasmid and cosmid clones. Bethesda Research Labs Focus 9 (1987) 12.

L. I. Patrushev, A. G. Valiaev, P. A. Golovchenko, S. V. Vinogradov, M. L. Chikindas, V. I. Kieselev. Cloning of the gene for thermostable Thermus aquaticus YT-1 DNA polymerase and its expression in Escherichia coli. Mol. Biol. (Mosk) 27 (1993) 1100-1112.

N. Gerald, I. Coppens, D. Dwyer. Molecular dissection and expression of the LdK39 kinesin in the human pathogen, Leishmania donovani. Molec. Microbio. 63 (4) (2007) 962-979.

S. Korolev, N. Murad, W. M. Barnes, E. DiCera, G. Waksman. Crystal structure of the large fragment of Thermus aquaticus DNA polymerase 1 at 2.5 A: Structural basis for thermostability. Proc. Natl. Acad. Sci. USA 92 (1995) 9264-9268.

S. C. Makrides. Strategies for achieving high-level expression of genes in Escherichia coli. Microbiol. Rev. 60 (1996) 512-538.

T. D. Brock, H. Freeze. Thermus aquaticus gen. n. and sp. n., a non-sporulating extreme thermophile. J. Bacteriol. 98 (1969) 289-297.

U. K. Laemmli. Cleavage of structural proteins during the assembly of the head of bacteriophage T4. Nature 227 (1970) 680-685.

W. M. Barnes. The fidelity of Taq polymerase catalyzing PCR is improved by an N-terminal deletion. Gene 112 (1992) 29-35.

1. An isolated DNA comprising a sequence of SEQ ID NO: 1.

2. A recombinant plasmid comprising an isolated DNA comprising a sequence of SEQ ID NO: 1.

3. A microorganism comprising DNA comprising a sequence of SEQ ID NO: 1.

4. A self-replicating nucleic acid molecule comprising:
a promoter;
at least one inducible repressor;
a high copy number origin of replication;
a sequence able to prevent transcription from said promoters from entering the region comprising the origin of replication; and
a multiple cloning site wherein at least one nucleic acid encoding a protein of interest may be cloned.

5. The self-replicating nucleic acid molecule of claim 4, wherein the promoter is a promoter of the bacteriophage lambda.

6. The self-replicating nucleic acid molecule of claim 5, wherein the promoter is a rightward promoter of bacteriophage lambda or a leftward promoter of bacteriophage lambda.

7. The self-replicating nucleic acid molecule of claim 4, wherein the at least one inducible repressor is a temperature-inducible repressor.

8. The self-replicating nucleic acid molecule of claim 4, wherein the at least one inducible repressor is a chemically-inducible repressor.

9. The self-replicating nucleic acid molecule of claim 4, wherein the at least one inducible repressor is a temperature and chemically-inducible repressor.

10. The self-replicating nucleic acid molecule of claim 9, wherein the temperature and chemically-inducible repressor is a lambda repressor λcI^{ts} ind⁺.

11. The self-replicating nucleic acid molecule of claim 4, wherein the molecule comprises a plasmid.

12. The self-replicating nucleic acid molecule of claim 4, wherein the molecule comprises a vector.

13. The self-replicating nucleic acid molecule of claim 12, wherein the vector is an expression vector.

14. The self-replicating nucleic acid molecule of claim 4, wherein the promoter is controlled by the repressor.

15. A method of producing at least one protein, comprising inducing expression of the at least one protein using a recombinant plasmid comprising an isolated DNA having a sequence of SEQ ID NO: 1, wherein inducing comprises temperature induction.

16. The method according to claim 15, wherein inducing further comprises chemical induction.

17. The method according to claim 15, wherein the recombinant plasmid comprising an isolated DNA having a sequence of SEQ ID NO: 1 further comprises at least one nucleic acid encoding the at least one protein.

18. A method of producing at least one protein, comprising inducing expression of the at least one protein using a recombinant plasmid comprising an isolated DNA having a sequence of SEQ ID NO: 1, wherein inducing comprises chemical induction.

19. The method according to claim 5, wherein inducing further comprises temperature induction.

20. A protein production system comprising:
a self-replicating nucleic acid molecule comprising:
a promoter of bacteriophage lambda;
a high copy number origin of replication;
a sequence able to prevent transcription from said promoters from entering the region comprising the origin of replication; and
a multiple cloning site; and
an inducible repressor located on a chromosome.

21. The protein production system of claim 20, wherein the promoter is the rightward promoter of bacteriophage lambda or the leftward promoter of bacteriophage lambda.

22. The system of claim 20, wherein the self-replicating molecule comprises a plasmid.

23. The system of claim 20, wherein the self-replicating molecule comprises an expression vector.

24. The system of claim 11, wherein the promoter is controlled by the repressor.

25. The system of claim 20, wherein the self-replicating nucleic acid molecule and the repressor are located in a living organism.

26. The system of claim 20, wherein the repressor is located on a host chromosome in the living organism.

27. The system of claim 20, wherein the repressor is a temperature inducible repressor.

28. The system of claim 20, wherein the repressor is a chemical inducible repressor.

29. The system of claim 20, wherein the repressor is a chemical inducible repressor and a temperature inducible repressor.

30. A protein production system comprising a self-replicating nucleic acid molecule comprising:
a promoter of bacteriophage lambda;
a high copy number origin of replication;
a sequence able to prevent transcription from said promoters from entering the region comprising the origin of replication;
a multiple cloning site; and
an inducible repressor.

31. The system of claim 30, wherein the repressor is a temperature inducible repressor.

32. The system of claim 30, wherein the repressor is a chemical inducible repressor.

33. The system of claim 30, wherein the repressor is a chemical inducible repressor and a temperature inducible repressor.

34. A method for protein purification comprising:
a) obtaining a cell lysate from a cell comprising DNA having a sequence of SEQ ID NO: 1;
b) treating the cell lysate with heat to denature cellular proteins;
c) precipitating and removing cellular DNA thereby obtaining a supernatant comprising the denatured cellular proteins;
d) applying the supernatant on a system of two chromatography columns, the first column comprising a cation-exchanger and the second column comprising an affinity-chromatography column; and
eluting the proteins,
thereby obtaining purified proteins.

35. An E. coli-based protein production system comprising:
an E. coli cell comprising a self-replicating nucleic acid molecule comprising:
a promoter of bacteriophage lambda;
a high copy number origin of replication;
a sequence able to prevent transcription from said promoters from entering the region comprising the origin of replication; and
a sequence encoding an LdK39 protein or fragment thereof.

36. The system of claim 35, wherein the LdK39 protein consist of LdK39-745.

37. The system of claim 35, wherein the nucleic acid molecule comprises pcl^{ts} ind⁺ LdK39-745.


SEQUENCE LISTING (1 sequence)

SEQ ID NO: 1 - protein, 5188 aa, Artificial Sequence
  Plasmid constructed from pcI plasmid, bacteriophage lambda regulatory sequences, and thermus aquatius DNA polymerase sequences
CATATGGGTAAACGTAAATCTACTGCCTTTCTGGAGAGGCTTGAGTTTGGCAGCCTCCTCCACGAGTTCGGCCTTCTGGAAAGCCCCAAGGCCCTGGAGGAGGCCCCCTGGCCCCCGCCGGAAGGGGCCTTCGTGGGCTTTGTGCTTTCCCGCAAGGAGCCCATGTGGGCCGATCTTCTGGCCCTGGCCGCCGCCAGGGGGGGCCGGGTCCACCGGGCCCCCGAGCCTTATAAAGCCCTCAGGGACCTGAAGGAGGCGCGGGGGCTTCTCGCCAAAGACCTGAGCGTTCTGGCCCTGAGGGAAGGCCTTGGCCTCCCGCCCGGCGACGACCCCATGCTCCTCGCCTACCTCCTGGACCCTTCCAACACCACCCCCGAGGGGGTGGCCCGGCGCTACGGCGGGGAGTGGACGGAGGAGGCGGGGGAGCGGGCCGCCCTTTCCGAGAGGCTCTTCGCCAACCTGTGGGGGAGGCTTGAGGGGGAGGAGAGGCTCCTTTGGCTTTACCGGGAGGTGGAGAGGCCCCTTTCCGCTGTCCTGGCCCACATGGAGGCCACGGGGGTGCGCCTGGACGTGGCCTATCTCAGGGCCTTGTCCCTGGAGGTGGCCGAGGAGATCGCCCGCCTCGAGGCCGAGGTCTTCCGCCTGGCCGGCCACCCCTTCAACCTCAACTCCCGGGACCAGCTGGAAAGGGTCCTCTTTGACGAGCTAGGGCTTCCCGCCATCGGCAAGACGGAGAAGACCGGCAAGCGCTCCACCAGCGCCGCCGTCCTGGAGGCCCTCCGCGAGGCCCACCCCATCGTGGAGAAGATCCTGCAGTACCGGGAGCTCACCAAGCTGAAGAGCACCTACATTGACCCCTTGCCGGACCTCATCCACCCCAGGACGGGCCGCCTCCACACCCGCTTCAACCAGACGGCCACGGCCACGGGCAGGCTAAGTAGCTCCGATCCCAACCTCCAGAACATCCCCGTCCGCACCCCGCTTGGGCAGAGGATCCGCCGGGCCTTCATCGCCGAGGAGGGGTGGCTATTGGTGGCCCTGGACTATAGCCAGATAGAGCTCAGGGTGCTGGCCCACCTCTCCGGCGACGAGAACCTGATCCGGGTCTTCCAGGAGGGGCGGGACATCCACACGGAGACCGCCAGCTGGATGTTCGGCGTCCCCCGGGAGGCCGTGGACCCCCTGATGCGCCGGGCGGCCAAGACCATCAACTTCGGGGTCCTCTACGGCATGTCGGCCCACCGCCTCTCCCAGGAGCTAGCCATCCCTTACGAGGAGGCCCAGGCCTTCATTGAGCGCTACTTTCAGAGCTTCCCCAAGGTGCGGGCCTGGATTGAGAAGACCCTGGAGGAGGGCAGGAGGCGGGGGTACGTGGAGACCCTCTTCGGCCGCCGCCGCTACGTGCCAGACCTAGAGGCCCGGGTGAAGAGCGTGCGGGAGGCGGCCGAGCGCATGGCCTTCAACATGCCCGTCCAGGGCACCGCCGCCGACCTCATGAAGCTGGCTATGGTGAAGCTCTTCCCCAGGCTGGAGGAAATGGGGGCCAGGATGCTCCTTCAGGTCCACGACGAGCTGGTCCTCGAGGCCCCAAAAGAGAGGGCGGAGGCCGTGGCCCGGCTGGCCAAGGAGGTCATGGAGGGGGTGTATCCCCTGGCCGTGCCCCTGGAGGTGGAGGTGGGGATAGGGGAGGACTGGCTCTCCGCCAAGGAGTGAGCATGCAGTAGGGAACTGCCAGGCATCAAATAAAACGAAAGGCTCAGTCGAAAGACTGGGCCTTTCGTTTTATCTGTTGTTTGTCGGTGAACGCTCTCCTGAGTAGGACAAATCCGCCGGGAGCGGATTTGAACGTTGCGAAGCAACGGCCCGGAGGGTGGCGGGCAGGACGCCCGCCATAAACTGCCAGGCATCAAATTAAGCAGAAGGCCATCCTGACGGATGGCCTTTTTGCGTTTCTACAAACTCTTTTTGTTTATTTTTCTAAATACATTCAAATATGTATCCGCTCATGAGACAATAGATCTAAGCTTGGCGTAATCATGGTCATAGCTGTTTCCTGTGTGAAATTGTTATCCGCTCACAATTCCACACAACATACGAGCCGGAAGCATAAAGTGTAAAGCCTGGGGTGCCTAATGAGTGAGCTAACTCACATTAATTGCGTTGCGCTCACTGCCCGCTTTCCAGTCGGGAAACCTGTCGTGCCAGCTGCATTAATGAATCGGCCAACGCGCGGGGAGAGGCGGTTTGCGTATTGGGCGCTCTTCCGCTTCCTCGCTCACTGACTCGCTGCGCTCGGTCGTTCGGCTGCGGCGAGCGGTATCAGCTCACTCAAAGGCGGTAATACGGTTATCCACAGAATCAGGGGATAACGCAGGAAAGAACATGTGAGCAAAAGGCCAGCAAAAGGCCAGGAACCGTAAAAAGGCCGCGTTGCTGGCGTTTTTCCATAGGCTCCGCCCCCCTGACGAGCATCACAAAAATCGACGCTCAAGTCAGAGGTGGCGAAACCCGACAGGACTATAAAGATACCAGGCGTTTCCCCCTGGAAGCTCCCTCGTGCGCTCTCCTGTTCCGACCCTGCCGCTTACCGGATACCTGTCCGCCTTTCTCCCTTCGGGAAGCGTGGCGCTTTCTCATAGCTCACGCTGTAGGTATCTCAGTTCGGTGTAGGTCGTTCGCTCCAAGCTGGGCTGTGTGCACGAACCCCCCGTTCAGCCCGACCGCTGCGCCTTATCCGGTAACTATCGTCTTGAGTCCAACCCGGTAAGACACGACTTATCGCCACTGGCAGCAGCCACTGGTAACAGGATTAGCAGAGCGAGGTATGTAGGCGGTGCTACAGAGTTCTTGAAGTGGTGGCCTAACTACGGCTACACTAGAAGAACAGTATTTGGTATCTGCGCTCTGCTGAAGCCAGTTACCTTCGGAAAAAGAGTTGGTAGCTCTTGATCCGGCAAACAAACCACCGCTGGTAGCGGTGGTTTTTTTGTTTGCAAGCAGCAGATTACGCGCAGAAAAAAAGGATCTCAAGAAGATCCTTTGATCTTTTCTACGGGGTCTGACGCTCAGTGGAACGAAAACTCACGTTAAGGGATTTTGGTCATGAGATTATCAAAAAGGATCTTCACCTAGATCCTTTTAAATTAAAAATGAAGTTTTAAATCAATCTAAAGTATATATGAGTAAACTTGGTCTGACAGTTACCAATGCTTAATCAGTGAGGCACCTATCTCAGCGATCTGTCTATTTCGTTCATCCATAGTTGCCTGACTCCCCGTCGTGTAGATAACTACGATACGGGAGGGCTTACCATCTGGCCCCAGTGCTGCAATGATACCGCGAGACCCACGCTCACCGGCTCCAGATTTATCAGCAATAAACCAGCCAGCCGGAAGGGCCGAGCGCAGAAGTGGTCCTGCAACTTTATCCGCCTCCATCCAGTCTATTAATTGTTGCCGGGAAGCTAGAGTAAGTAGTTCGCCAGTTAATAGTTTGCGCAACGTTGTTGCCATTGCTACAGGCATCGTGGTGTCACGCTCGTCGTTTGGTATGGCTTCATTCAGCTCCGGTTCCCAACGATCAAGGCGAGTTACATGATCCCCCATGTTGTGCAAAAAAGCGGTTAGCTCCTTCGGTCCTCCGATCGTTGTCAGAAGTAAGTTGGCCGCAGTGTTATCACTCATGGTTATGGCAGCACTGCATAATTCTCTTACTGTCATGCCATCCGTAAGATGCTTTTCTGTGACTGGTGAGTACTCAACCAAGTCATTCTGAGAATAGTGTATGCGGCGACCGAGTTGCTCTTGCCCGGCGTCAACACGGGATAATACCGCGCCACATAGCAGAACTTTAAAAGTGCTCATCATTGGAAAACGTTCTTCGGGGCGAAAACTCTCAAGGATCTTACCGCTGTTGAGATCCAGTTCGATGTAACCCACTCGTGCACCCAACTGATCTTCAGCATCTTTTACTTTCACCAGCGTTTCTGGGTGAGCAAAAACAGGAAGGCAAAATGCCGCAAAAAAGGGAATAAGGGCGACACGGAAATGTTGAATACTCATACTCTTCCTTTTTCAATATTATGTAAGCAGACAGTTTTATTGTTCATGATGATATATTTTTATCTTGTGCAATGTAACATCAGAGATTTTGAGACACAACGTGGCTTTGTTGAATAAATCGAACTTTTGCTGAGTTGACTCCCCGCGCGGACATTAATTGCGTTGCGCTCACTGCCCGCTTTCCAGTCGGGAAACCTGTCGTGCCAGCTGCATTAATGAATCGGCCAACGCGCGGGGAGAGGCGGTTTGCGTATTGGGCGCCATAGACGTCTTTGAATTGTTATCAGCTATGCGCCGACCAGAACACCTTGCCGATCAGCCAAACGTCTCTTCAGGCCACTGACTAGCGATAACTTTCCCCACAACGGAACAACTCTCATTGCATGGGATCATTGGGTACTGTGGGTTTAGTGGTTGTAAAAACACCTGACCGCTATCCCTGATCAGTTTCTTGAAGGTAAACTCATCACCCCCAAGTCTGGCTATGCAGAAATCACCTGGCTCAACAGCCTGCTCAGGGTCAACGAGAATTAACATTCCGTCAGGAAAGCTTGGCTTGGAGCCTGTTGGTGCGGTCATGGAATTACCTTCAACCTCAAGCCAGAATGCAGAATCACTGGCTTTTTTGGTTGTGCTTACCCATCTCTCCGCATCACCTTTGGTAAAGGTTCTAAGCTCAGGTGAGAACATCCCTGCCTGAACATGAGAAAAAACAGGGTACTCATACTCACTTCTAAGTGACGGCTGCATACTAACCGCTTCATACATCTCGTAGATTTCTCTGGCGATTGAAGGGCTAAATTCTTCAACGCTAACTTTGAGAATTTTTGTAAGCAATGCGGCGTTATAAGCATTTAATGCATTGATGCCATTAAATAAAGCACCAACGCCTGACTGCCCCATCCCCATCTTGTCTGCGACAGATTCCTGGGATAAGCCAAGTTCATTTTTCTTTTTTTCATAAATTGCTTTAAGGCGACGTGCGTCCTCAAGCTGCTCTTGTGTTAATGGTTTCTTTTTTGTGCTCATACGTTAAATCTATCACCGCAAGGGATAAATATCTAACACCGTGCGTGTTGACTATTTTACCTCTGGCGGTGATAATGGTTGCATGTACTAAGGAGGTT